(19) United States
(12) Patent Application Publication
(10) Pub. No.: US 2026/0285958 A1
(43) Pub. Date: Sep. 24, 2026

(54) Compositions and Methods Related to DKK1 Binders

(71) Applicant: Twist Bioscience Corporation, South San Francisco, CA (US)

(72) Inventors: Aaron Sato, Burlingame, CA (US); Linya Wang, Milpitas, CA (US)

(73) Assignee: Twist Bioscience Corporation, South San Francisco, CA (US)

(21) Appl. No.: 19/473,898

(22) PCT Filed: Apr. 12, 2024
(86) PCT No.: PCT/US2024/024316

§ 371 Date: Oct. 9, 2025

Related U.S. Application Data
(60) Provisional application No. 63/496,315, filed on Apr. 14, 2023.
(60) Provisional application No. 63/631,427, filed on Apr. 8, 2024.

(51) Int. Cl. C07K 16/18; A61K 39/00; A61P 35/00

(52) U.S. Cl. CPC C07K 16/18; A61P 35/00; A61K 2039/505; C07K 2317/31; C07K 2317/52; C07K 2317/565; C07K 2317/569; C07K 2317/92

(57) ABSTRACT

Provided herein are compositions relating to Dickkopf WNT signaling pathway inhibitor 1 (DKK1) binders, including bi-specific DKK1 binders. Further described herein are methods of making and using the DKK1 binders.

### CROSS-REFERENCE TO RELATED APPLICATIONS

This application claims the benefit of priority of U.S. Provisional Application No. 63/496,315, filed Apr. 14, 2023, and U.S. Provisional Application No. 63/631,427, filed Apr. 8, 2024, each of which is incorporated by reference herein it its entirety for any purpose.

### SEQUENCE LISTING

The present application contains a Sequence Listing, which has been submitted electronically in XML format and is hereby incorporated by reference in its entirety. Said XML copy, created on Apr. 12, 2024, is named “01356-0032-00PCT.xml” and is 29,023 bytes in size. The information in the electronic format of the sequence listing is incorporated herein by reference in its entirety.

### BACKGROUND

Dickkopf WNT signaling pathway inhibitor 1 (also known as dickkopf-1 or DKK1) is a secreted glycoprotein characterized by two cysteine-rich domains that mediate protein-protein interactions. DKK1 is involved in embryonic development of the heart, head, and forelimbs through its inhibition of the WNT signaling pathway. In adults, elevated expression of this gene has been observed in numerous human cancers, and this protein may promote proliferation, invasion, and growth in cancer cell lines. Given the role of DKK1 in various diseases and disorders, there is a need for improved therapeutics.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Provided herein are polypeptides that bind DKK1 comprising: a first antibody variable domain comprising a CDR1 comprising the amino acid of SEQ ID NO: 11 or 17, a CDR2 comprising the amino acid sequence of SEQ ID NO: 12 or 18, and a CDR3 comprising the amino acid sequence of SEQ ID NO: 13 or 19; and a second antibody variable domain comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, (a) (i) the first antibody variable domain comprises a CDR1 comprising the amino acid of SEQ ID NO: 11, a CDR2 comprising the amino acid sequence of SEQ ID NO: 12, and a CDR3 comprising the amino acid sequence of SEQ ID NO: 13; and (ii) the second antibody variable domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or (b) (i) the first antibody variable domain comprises a CDR1 comprising the amino acid of SEQ ID NO: 17, a CDR2 comprising the amino acid sequence of SEQ ID NO: 18, and a CDR3 comprising the amino acid sequence of SEQ ID NO: 19; and (ii) the second antibody variable domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, the first antibody variable domain comprises an amino acid sequence at least 80%, at least 85%, at least 90%, at least 95%, at least 96%, at least 97%, at least 98%, or at least 99% identical to the amino acid sequence of SEQ ID NO: 22 or SEQ ID NO: 23. In some embodiments, the first antibody variable domain comprises the amino acid sequence of SEQ ID NO: 22 or SEQ ID NO: 23.

In some embodiments, the second antibody variable domain comprises an amino acid sequence at least 80%, at least 85%, at least 90%, at least 95%, at least 96%, at least 97%, at least 98%, or at least 99% identical to the amino acid sequence of the amino acid sequence of SEQ ID NO: 24. In some embodiments, the polypeptide of any one of claims 1 to 5, wherein the second antibody variable domain comprises the amino acid sequence of SEQ ID NO: 24.

In some embodiments, the polypeptide comprises an Fc region. In some embodiments, the Fc region is an IgG1, IgG2, IgG3, or IgG4 Fc region. In some embodiments, the polypeptide has the structure: first antibody variable domain-linker-Fc region-linker-second antibody variable domain.

In some embodiments, polypeptide comprises an amino acid sequence at least 80%, at least 85%, at least 90%, at least 95%, at least 96%, at least 97%, at least 98%, or at least 99% identical to the amino acid sequence of SEQ ID NO: 3 or SEQ ID NO: 4. In some embodiments, the polypeptide comprises the amino acid sequence of SEQ ID NO: 3 or SEQ ID NO: 4.

Also provided herein are peptides that bind DKK1 comprising a CDR1 comprising the amino acid of SEQ ID NO: 5, a CDR2 comprising the amino acid sequence of SEQ ID NO: 6, and a CDR3 comprising the amino acid sequence of SEQ ID NO: 7.

In some embodiments, the polypeptide comprises an amino acid sequence at least 80%, at least 85%, at least 90%, at least 95%, at least 96%, at least 97%, at least 98%, or at least 99% identical to the amino acid sequence of SEQ ID NO: 20. In some embodiments, the polypeptide comprises the amino acid sequence of SEQ ID NO: 20.

In some embodiments, the polypeptide comprises an Fc region. In some embodiments, the Fc region is an IgG1, IgG2, IgG3, or IgG4 Fc region.

In some embodiments, the polypeptide comprises an amino acid sequence at least 80%, at least 85%, at least 90%, at least 95%, at least 96%, at least 97%, at least 98%, or at least 99% identical to the amino acid sequence of SEQ ID NO: 1. In some embodiments, the polypeptide comprises SEQ ID NO: 1.

Also provided herein are polypeptides that bind DKK1 comprising an amino acid sequence at least 80% identical to any one of SEQ ID NOs: 1-4. In some embodiments, the polypeptide comprises an amino acid sequence at least 85% identical to any one of SEQ ID NOs: 1-4. In some embodiments, the polypeptide comprises an amino acid sequence at least 90% identical to any one of SEQ ID NOs: 1-4. In some embodiments, the polypeptide comprises an amino acid sequence at least 95% identical to any one of SEQ ID NOs: 1-4. In some embodiments, the polypeptide comprises an amino acid sequence of at least 96%, at least 97%, at least 98%, or at least 99% identical to any one of SEQ ID NOs: 1-4. In some embodiments, the polypeptide comprises an amino acid sequence of any one of SEQ ID NOs: 1-4.

In some embodiments, the polypeptide is a monoclonal antibody, a bi-specific antibody, a multispecific antibody, a grafted antibody, a humanized antibody, a synthetic antibody, a chimeric antibody, a single chain antibody, a single-domain antibody, a diabody, a fragment comprised of only a single monomeric variable domain, an intrabody, or antigen-binding fragments thereof. In some embodiments, the polypeptide is a single domain antibody.

In some embodiments, the polypeptide binds to DKK1. In some embodiments, the polypeptide binds to DKK1 with a KD of less than 50 nM. In some embodiments, the polypeptide binds to DKK1 with a KD of less than 25 nM. In some embodiments, the polypeptide binds to DKK1 with a KD of less than 10 nM. In some embodiments, the polypeptide binds to DKK0-1 with a KD of less than 5 nM.

Also provided herein are pharmaceutical compositions comprising a polypeptide provided herein and a pharmaceutically acceptable carrier.

Further provided herein are isolated nucleic acids that encode any of the polypeptides described herein. Also provided are host cells comprising the nucleic acids that encode any of the polypeptides described herein.

Also provided herein are vectors comprising nucleic acids that encode any of the polypeptides described herein. Also provided are host cells comprising the vectors comprising nucleic acids that encode any of the polypeptides described herein.

Further provided herein are methods of producing any of the polypeptides described herein comprising incubating a host cell comprising a nucleic acid that encodes the polypeptide under conditions suitable for expression of the polypeptide. Also provided herein are methods of producing any of the polypeptides described herein comprising incubating a host cell comprising a vector comprising a nucleic acid that encodes the polypeptide under conditions suitable for expression of the polypeptide. In some embodiments, the method further comprises isolating the polypeptide.

Also provided herein are methods for treating a subject having a cancer with elevated expression of DKK1 comprising administering to the subject a pharmaceutically effective amount of any of the polypeptide described herein or a pharmaceutical composition thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A depicts a first schematic of an immunoglobulin.

FIG. 1B depicts a second schematic of an immunoglobulin.

FIG. 2 depicts a schematic of a motif for placement in an immunoglobulin.

FIG. 3A depicts a schematic of an immunoglobulin comprising a VH domain attached to a VL domain using a linker.

FIG. 3B depicts a schematic of a full-domain architecture of an immunoglobulin comprising a VH domain attached to a VL domain using a linker, a leader sequence, and pIII sequence.

FIG. 3C depicts a schematic of four framework elements (FW1, FW2, FW3, FW4) and the variable 3 CDR (L1, L2, L3) elements for a VL or VH domain.

FIGS. 4A-4B shows the process for selection of anti-DKK1 antibodies from a library of 109 sequences.

FIG. 5A depicts epitope binning of DKK1 binders. Two epitope bins were apparent among the DKK1 binders. Formation of Ab-Ag-Ab complex indicates the antibodies are not binding to the same epitope of DKK1.

FIG. 5B depicts anti-DKK1 antibody affinity for hDKK1 CRD1, hDKK1 CRD2, hDKK1, and cynomolgus monkey DKK1. DKK1-5, DKK1-13, and DKK1-14 correspond to DKK1-97, DKK1-98, and DKK1-94, respectively, of WO 2023/091614. DKK1-6 and DKK1-7 correspond to DKK1-37 and DKK1-10, respectively, of WO 2023/091614.

FIG. 6 depicts Wnt TCF/LEF reporter assay screening. Wnt TCF/LEF signaling was blocked by DKK1 binding to LRP5/6. Screening of DKK1 binders for blocking DKK1 binding, resulting in restoration of Wnt signaling for those binding to DKK1 CRD2.

FIG. 7 depicts in vitro primary immune cell activation. DKK1 leads to immune suppression including T cell inactivation, MDSC accumulation, and NK cell clearance. Human PBMCs were treated with immune stimulator, Wnt3a, hDKK1, and DKK mAbs. Cytokine release of GM-CSF, markers for NK cell activation, was measured by ELISA. GM-CSF is the markers for NK cell activation. Antibodies binding to CDR1 of DKK1 showed stronger NK cell activation.

FIGS. 8A-8B depict in vitro PC3 tumor cytotoxicity by activated immune cells. FIG. 8A: T cells and NK cells in human PBMC were activated and co-cultured with PC3 tumor cells for 6 days. Activated immune cells kill PC3 tumor cells, while hDKK1 treatment inhibits cytotoxicity.

FIG. 8B: Blocking the interaction of hDKK1 to the receptor with DKK1 binders restores the cytotoxicity potency.

FIGS. 9A-9C depict a BsAb functional assay. DKK1-13 and DKK1-14 bind to DKK1 CRD1 and activate immune response, while DKK1-5 binds to DKK1 CRD2 and activates Wnt signaling. FIG. 9A depicts Wnt TCF/LEF reporter analysis of bi-specific Abs DKK1-135 and DKK1-136 compared to DKK1-13, DKK1-14, and DKK1-5. FIG. 9B-9C show the potency of activating both Wnt (FIG. 9B) and immune response (FIG. 9C).

FIGS. 10A-10D depict anti-DKK1 binders in a tumor regression model. FIG. 10A depicts that homozygous SCID mice were inoculated with PC3 cells. Dosing was initiated at tumor volume average of ~100 mm3 with 10 mg/kg via intraperitoneal injection once every 3 days for 8 cycles (Q3Dx8). Tumor sizes were measured 3 times a week. FIGS. 10B-10D depict that anti-DKK1 treatment downregulates tumor growth, showing its efficacy in tumor suppression.

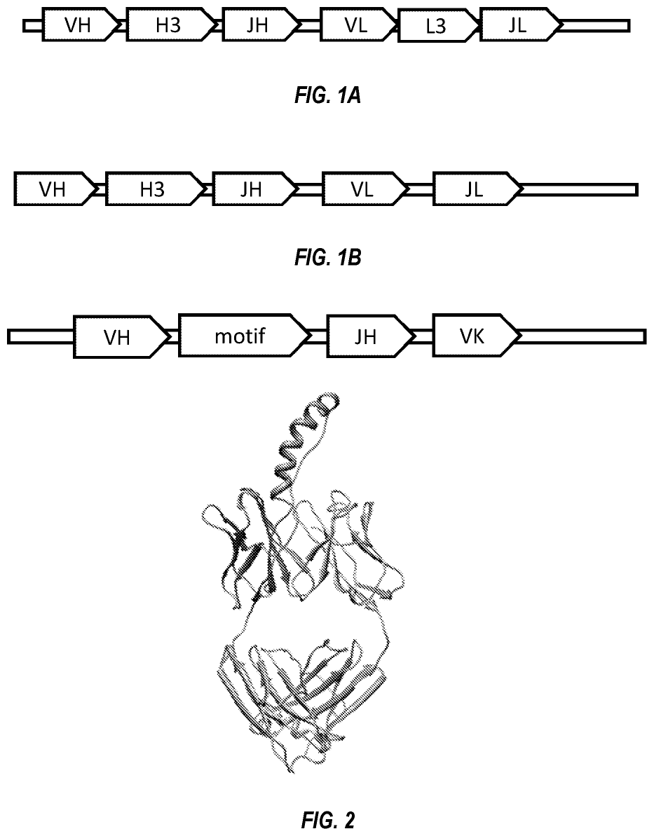

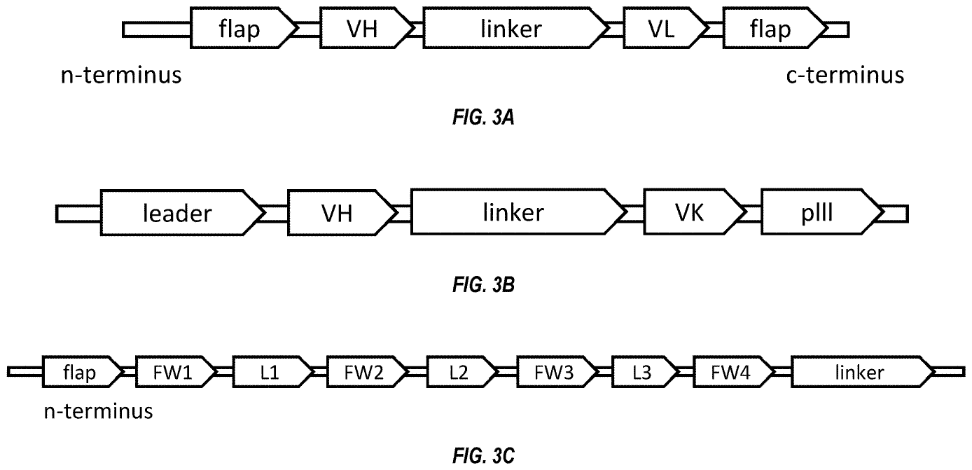

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

The section headings used herein are for organizational purposes only and are not to be construed as limiting the subject matter described.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, components, and/or groups, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

Unless specifically stated, as used herein, the term “nucleic acid” encompasses double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). Nucleic acid sequences, when provided, are listed in the 5′ to 3′ direction, unless stated otherwise. Methods described herein provide for the generation of isolated nucleic acids. Methods described herein additionally provide for the generation of isolated and purified nucleic acids. A “nucleic acid” as referred to herein can comprise at least 5, 10, 20, 30, 40, 50, 60, 70, 80, 90, 100, 125, 150, 175, 200, 225, 250, 275, 300, 325, 350, 375, 400, 425, 450, 475, 500, 600, 700, 800, 900, 1000, 1100, 1200, 1300, 1400, 1500, 1600, 1700, 1800, 1900, 2000, or more bases in length. Moreover, provided herein are methods for the synthesis of any number of polypeptide-segments encoding nucleotide sequences, including sequences encoding non-ribosomal peptides (NRPs), sequences encoding non-ribosomal peptide-synthetase (NRPS) modules and synthetic variants, polypeptide segments of other modular proteins, such as antibodies, polypeptide segments from other protein families, including non-coding DNA or RNA, such as regulatory sequences e.g. promoters, transcription factors, enhancers, siRNA, shRNA, RNAi, miRNA, small nucleolar RNA derived from microRNA, or any functional or structural DNA or RNA unit of interest. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. cDNA encoding for a gene or gene fragment referred herein may comprise at least one region encoding for exon sequences without an intervening intron sequence in the genomic equivalent sequence.

Unless otherwise defined, scientific and technical terms used in connection with the present disclosure shall have the meanings that are commonly understood by those of ordinary skill in the art. Further, unless otherwise required by context or expressly indicated, singular terms shall include pluralities and plural terms shall include the singular. For any conflict in definitions between various sources or references, the definition provided herein will control.

In general, the numbering of the residues in an antibody heavy chain is that of the EU index as in Kabat et al., Sequences of Proteins of Immunological Interest, 5th Ed. Public Health Service, National Institutes of Health, Bethesda, Md. (1991). Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The terms “polypeptide” and “protein” are used interchangeably to refer to a polymer of amino acid residues, and are not limited to a minimum length. Such polymers of amino acid residues may contain natural or non-natural amino acid residues, and include, but are not limited to, peptides, oligopeptides, dimers, trimers, and multimers of amino acid residues. Both full-length proteins and fragments thereof are encompassed by the definition. The terms also include post-expression modifications of the polypeptide, for example, glycosylation, sialylation, acetylation, phosphorylation, and the like. Furthermore, for purposes of the present disclosure, a “polypeptide” refers to a protein which includes modifications, such as deletions, additions, and substitutions (generally conservative in nature), to the native sequence, as long as the protein maintains the desired activity. These modifications may be deliberate, as through site-directed mutagenesis, or may be accidental, such as through mutations of hosts which produce the proteins or errors due to PCR amplification.

The terms “DKK1,” “dickkopf WNT signaling pathway inhibitor 1,” “Dickkopf-1,” “Dkk-1,” “hDkk-1,” and “SK” as used herein refer to any native, mature DKK1 that results from processing of a DKK1 precursor in a cell. The term includes DKK1 from any vertebrate source, including mammals such as primates (e.g., humans and cynomolgus or rhesus monkeys) and rodents (e.g., mice and rats), unless otherwise indicated. The term also includes naturally-occurring variants of DKK1, such as splice variants or allelic variants. A nonlimiting exemplary precursor human DKK1 amino acid sequence is shown, e.g., in UniProtKB/Swiss-Prot Accession: 094907.1. See SEQ ID NO: 25 (signal sequence italicized; CRD1 and CRD2 underlined).

(SEQ ID NO: 25)
MMALGAAGAT RVFVAMVAAA LGGHPLLGVS
ATLNSVLNSN AIKNLPPPLG GAAGHPGSAV
SAAPGILYPG GNKYQTIDNY QPYPCAEDEE
CGTDEYCASP TRGGDAGVQI CLACRKRRKR
CMRHAMCCPG NYCKNGICVS SDONHERGEI
EETITESEGN DHSTLDGYSR RTTLSSKMYH
TKGQEGSVCL RSSDCASGLC CARHEWSKIC
KPVLKEGQVC TKHRRKGSHG LEIFQRCYCG
EGLSCRIQKD HHQASNSSRL HTCQRH.

The term “specifically binds” to an antigen or epitope is a term that is well understood in the art, and methods to determine such specific binding are also well known in the art. A molecule is said to exhibit “specific binding” or “preferential binding” if it reacts or associates more frequently, more rapidly, with greater duration and/or with greater affinity with a particular cell or substance than it does with alternative cells or substances. A single-domain antibody (sdAb) or VHH-containing polypeptide “specifically binds” or “preferentially binds” to a target if it binds with greater affinity, avidity, more readily, and/or with greater duration than it binds to other substances. For example, a sdAb or VHH-containing polypeptide that specifically or preferentially binds to a DKK1 epitope is a sdAb or VHH-containing polypeptide that binds this epitope with greater affinity, avidity, more readily, and/or with greater duration than it binds to other DKK1 epitopes or non-DKK1 epitopes. It is also understood by reading this definition that; for example, a sdAb or VHH-containing polypeptide that specifically or preferentially binds to a first target may or may not specifically or preferentially bind to a second target. As such, “specific binding” or “preferential binding” does not necessarily require (although it can include) exclusive binding. Generally, but not necessarily, reference to binding means preferential binding. “Specificity” refers to the ability of a binding protein to selectively bind an antigen.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In some embodiments, by “reduce” or “inhibit” is meant the ability to cause an overall decrease of 10% or greater. In some embodiments, by “reduce” or “inhibit” is meant the ability to cause an overall decrease of 50% or greater. In some embodiments, by “reduce” or “inhibit” is meant the ability to cause an overall decrease of 75%, 85%, 90%, 95%, or greater. In some embodiments, the amount noted above is inhibited or decreased over a period of time, relative to a control over the same period of time.

The term “antibody” is used in the broadest sense and encompass various polypeptides that comprise antibody-like antigen-binding domains, including but not limited to conventional antibodies (typically comprising at least one heavy chain and at least one light chain), single-domain antibodies (sdAbs, comprising at least one VHH domain and an Fc region), VHH-containing polypeptides (polypeptides comprising at least one VHH domain), and fragments of any of the foregoing so long as they exhibit the desired antigen-binding activity. In some embodiments, an antibody comprises a dimerization domain. Such dimerization domains include, but are not limited to, heavy chain constant domains (comprising CH1, hinge, CH2, and CH3 domains, where CH1 typically pairs with a light chain constant domain, CL, and where the hinge mediates dimerization) and Fc regions (comprising hinge, CH2, and CH3, where the hinge mediates dimerization).

The term “antigen-binding domain” as used herein refers to a portion of an antibody sufficient to bind an antigen. In some embodiments, an antigen binding domain of a conventional antibody comprises three heavy chain CDRs and three light chain CDRs. Thus, in some embodiments, an antigen binding domain comprises a heavy chain variable region comprising CDR1-FR2-CDR2-FR3-CDR3, and any portions of FR1 and/or FR4 required to maintain binding to antigen, and a light chain variable region comprising CDR1-FR2-CDR2-FR3-CDR3, and any portions of FR1 and/or FR4 required to maintain binding to antigen. In some embodiments, an antigen-binding domain of an sdAb or VHH-containing polypeptide comprises three CDRs of a VHH domain (e.g., CDR1, CDR2, and CDR3). Thus, in some embodiments, an antigen binding domain of an sdAb or VHH-containing polypeptide comprises a VHH domain comprising CDR1-FR2-CDR2-FR3-CDR3, and any portions of FR1 and/or FR4 required to maintain binding to antigen.

The term “VHH” or “VHH domain” or “VHH antigen-binding domain” as used herein refers to the antigen-binding portion of a single-domain antibody, such as a camelid antibody or shark antibody. In some embodiments, a VHH comprises three CDRs and four framework regions, designated FR1, CDR1, FR2, CDR2, FR3, CDR3, and FR4. In some embodiments, a VHH may be truncated at the N-terminus or C-terminus such that it comprises only a partial FR1 and/or FR4, or lacks one or both of those framework regions, so long as the VHH substantially maintains antigen binding and specificity.

The terms “single domain antibody” and “sdAb” are used interchangeably herein to refer to an antibody comprising at least one monomeric domain, such as a VHH domain, without a light chain, and an Fc region. In some embodiments, an sdAb is a dimer of two polypeptides wherein each polypeptide comprises at least one VHH domain and an Fc region. As used herein, the terms “single domain antibody” and “sdAb” encompass polypeptides that comprise multiple VHH domains, such as a polypeptide having the structure VHH₁-Fc-VHH₂, wherein VHH₁ and VHH₂ may be the same or different.

The term “VHH-containing polypeptide” refers to a polypeptide that comprises at least one VHH domain. In some embodiments, a VHH polypeptide comprises two, three, or four or more VHH domains, wherein each VHH domain may be the same or different. In some embodiments, a VHH-containing polypeptide comprises an Fc region. In some such embodiments, the VHH-containing polypeptide may be referred to as an sdAb. Further, in some such embodiments, the VHH polypeptide may form a dimer. Nonlimiting structures of VHH-containing polypeptides, which are also sdAbs, include VHH₁-Fc and VHH₁-Fc-VHH₂, and VHH₁-VHH₂-Fc, wherein VHH₁ and VHH₂ may be the same or different. In some embodiments of such structures, one VHH may be connected to another VHH by a linker, or one VHH may be connected to the Fc by a linker. In some such embodiments, the linker comprises 1-20 amino acids, preferably 1-20 amino acids predominantly composed of glycine and, optionally, serine and/or alanine. In some embodiments, when a VHH-containing polypeptide comprises an Fc, it forms a dimer. Thus, the structure VHH₁-Fc-VHH₂, if it forms a dimer, is considered to be tetravalent (i.e., the dimer has four VHH domains).

The term “monoclonal antibody” refers to an antibody (including an sdAb or VHH-containing polypeptide) of a substantially homogeneous population of antibodies, that is, the individual antibodies comprising the population are identical except for possible naturally-occurring mutations that may be present in minor amounts.

The term “CDR” denotes a complementarity determining region as defined by at least one manner of identification to one of skill in the art. In some embodiments, CDRs can be defined in accordance with any of the Chothia numbering schemes, the Kabat numbering scheme, a combination of Kabat and Chothia, the AbM definition, and/or the contact definition. A VHH comprises three CDRs, designated CDR1, CDR2, and CDR3.

The term “heavy chain constant region” as used herein refers to a region comprising at least three heavy chain constant domains, CH1, hinge, CH2, and CH3. Of course, non-function-altering deletions and alterations within the domains are encompassed within the scope of the term “heavy chain constant region,” unless designated otherwise. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an a constant region is an IgA antibody. Further, an antibody comprising a μ constant region is an IgM antibody, and an antibody comprising an F constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgAQ1 (comprising an ai constant region) and IgA2 (comprising an α₂ constant region) antibodies; and IgM antibodies include, but are not limited to, IgM1 and IgM2.

A “Fc region” as used herein refers to a portion of a heavy chain constant region comprising CH2 and CH3. In some embodiments, an Fc region comprises a hinge, CH2, and CH3. In various embodiments, when an Fc region comprises a hinge, wherein the hinge mediates dimerization between two Fc-containing polypeptides. An Fc region may be of any antibody heavy chain constant region isotype discussed herein. In some embodiments, an Fc region is an IgG1, IgG2, IgG3, or IgG4.

An “acceptor human framework” as used herein is a framework comprising the amino acid sequence of a heavy chain variable domain (VH) framework derived from a human immunoglobulin framework or a human consensus framework, as discussed herein. An acceptor human framework derived from a human immunoglobulin framework or a human consensus framework can comprise the same amino acid sequence thereof, or it can contain amino acid sequence changes. In some embodiments, the number of amino acid changes are fewer than 10, or fewer than 9, or fewer than 8, or fewer than 7, or fewer than 6, or fewer than 5, or fewer than 4, or fewer than 3, across all the human frameworks in a single antigen binding domain, such as a VHH.

“Affinity” refers to the strength of the sum total of noncovalent interactions between a single binding site of a molecule (for example, an antibody, such as an sdAb, or VHH-containing polypeptide) and its binding partner (for example, an antigen). The affinity or the apparent affinity of a molecule X for its partner Y can generally be represented by the dissociation constant (K_{D}) or the K_{D-apparent}, respectively. Affinity can be measured by common methods known in the art (such as, for example, ELISA K_{D}, KinExA, flow cytometry, and/or surface plasmon resonance (SPR) devices), including those described herein. Such methods include, but are not limited to, methods involving BIAcore®, Octet®, or flow cytometry.

The term “K_{D}”, as used herein, refers to the equilibrium dissociation constant of an antigen-binding molecule/antigen interaction. When the term “K_{D}” is used herein, it includes K_{D} and K_{D-apparent}.

A “humanized VHH” as used herein refers to a VHH in which one or more framework regions have been substantially replaced with human framework regions. In some instances, certain framework region (FR) residues of the human immunoglobulin are replaced by corresponding non-human residues. Furthermore, the humanized VHH can comprise residues that are found neither in the original VHH nor in the human framework sequences, but are included to further refine and optimize sdAb VHH-containing polypeptide performance. In some embodiments, a humanized sdAb or VHH-containing polypeptide comprises a human Fc region. As will be appreciated, a humanized sequence can be identified by its primary sequence and does not necessarily denote the process by which the antibody was created.

An “effector-positive Fc region” possesses an “effector function” of a native sequence Fc region. Exemplary “effector functions” include Fc receptor binding; Clq binding and complement dependent cytotoxicity (CDC); Fc receptor binding; antibody-dependent cell-mediated cytotoxicity (ADCC); phagocytosis; down regulation of cell surface receptors (for example B-cell receptor); and B-cell activation, etc. Such effector functions generally require the Fc region to be combined with a binding domain (for example, an antibody variable domain) and can be assessed using various assays.

A “variant Fc region” comprises an amino acid sequence which differs from that of a native sequence Fc region by virtue of at least one amino acid modification. In some embodiments, a “variant Fc region” comprises an amino acid sequence which differs from that of a native sequence Fc region by virtue of at least one amino acid modification, yet retains at least one effector function of the native sequence Fc region. In some embodiments, the variant Fc region has at least one amino acid substitution compared to a native sequence Fc region or to the Fc region of a parent polypeptide, for example, from about one to about ten amino acid substitutions, and preferably, from about one to about five amino acid substitutions in a native sequence Fc region or in the Fc region of the parent polypeptide. In some embodiments, the variant Fc region herein will possess at least about 80% sequence identity with a native sequence Fc region and/or with an Fc region of a parent polypeptide, at least about 90% sequence identity therewith, at least about 95%, at least about 96%, at least about 97%, at least about 98%, or at least about 99% sequence identity therewith.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an polypeptide of interest and the products screened for a desired activity, for example, retained/improved antigen binding, decreased immunogenicity, or improved ADCC or CDC.

TABLE 1
Amino acid residues

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term “vector” is used to describe a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase). The term “expression vector” refers to a vector that is used to express a polypeptide of interest in a host cell.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E, CHO-DG44, CHO-K1, CHO-S, and CHO-DS cells. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) a provided herein.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The terms “individual” and “subject” are used interchangeably herein to refer to an animal; for example, a mammal. In some embodiments, methods of treating mammals, including, but not limited to, humans, rodents, and simians, are provided. In some examples, an “individual” or “subject” refers to an individual or subject in need of treatment for a disease or disorder. In some embodiments, the subject to receive the treatment can be a patient, designating the fact that the subject has been identified as having a disease or disorder of relevance to the treatment, or being at adequate risk of contracting the disease or disorder.

A “disease” or “disorder” as used herein refers to a condition where treatment is needed and/or desired.

The terms “cancer” and “tumor” encompass solid and hematological/lymphatic cancers and also encompass malignant, pre-malignant, and benign growth, such as dysplasia.

As used herein, “treatment” is an approach for obtaining beneficial or desired clinical results. “Treatment” as used herein, covers any administration or application of a therapeutic for disease in a mammal, including a human. For purposes of this disclosure, beneficial or desired clinical results include, but are not limited to, any one or more of: alleviation of one or more symptoms, diminishment of extent of disease, preventing or delaying spread (for example, metastasis) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease. The methods provided herein contemplate any one or more of these aspects of treatment. In-line with the above, the term treatment does not require one-hundred percent removal of all aspects of the disorder.

A “therapeutically effective amount” of a substance/molecule, agonist or antagonist may vary according to factors such as the disease state, age, sex, and weight of the individual, and the ability of the substance/molecule, agonist or antagonist to elicit a desired response in the individual. A therapeutically effective amount is also one in which any toxic or detrimental effects of the substance/molecule, agonist or antagonist are outweighed by the therapeutically beneficial effects. A therapeutically effective amount may be delivered in one or more administrations. A therapeutically effective amount refers to an amount effective, at dosages and for periods of time necessary, to achieve the desired therapeutic and/or prophylactic result.

The terms “pharmaceutical formulation” and “pharmaceutical composition” are used interchangeably and refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and are compatible with other ingredients of the formulation. The pharmaceutically acceptable carrier is appropriate for the formulation employed.DKK1 Libraries

Provided herein are methods and compositions relating to dickkopf WNT signaling pathway inhibitor 1 (DKK1) variant immunoglobulins (e.g., antibody, VHH) comprising nucleic acids encoding for an immunoglobulin comprising a DKK1 binding domain. Immunoglobulins as described herein can stably support a DKK1 binding domain. Libraries as described herein may be further variegated to provide for variant libraries comprising nucleic acids each encoding for a predetermined variant of at least one predetermined reference nucleic acid sequence. Further described herein are protein libraries that may be generated when the nucleic acid libraries are translated. In some instances, nucleic acid libraries as described herein are transferred into cells to generate a cell library. Also provided herein are downstream applications for the libraries synthesized using methods described herein. Downstream applications include identification of variant nucleic acids or protein sequences with enhanced biologically relevant functions, e.g., improved stability, affinity, binding, functional activity, and for the treatment or prevention of a disease state associated with DKK1.

Provided herein are libraries comprising nucleic acids encoding for an immunoglobulin. In some instances, the immunoglobulin is an antibody. As used herein, the term antibody will be understood to include proteins having the characteristic two-armed, Y-shape of a typical antibody molecule as well as one or more fragments of an antibody that retain the ability to specifically bind to an antigen. Exemplary antibodies include, but are not limited to, a monoclonal antibody, a polyclonal antibody, a bi-specific antibody, a multispecific antibody, a grafted antibody, a human antibody, a humanized antibody, a synthetic antibody, a chimeric antibody, a camelized antibody, a single-chain Fvs (scFv) (including fragments in which the VL and VH are joined using recombinant methods by a synthetic or natural linker that enables them to be made as a single protein chain in which the VL and VH regions pair to form monovalent molecules, including single chain Fab and scFab), a single chain antibody, a Fab fragment (including monovalent fragments comprising the VL, VH, CL, and CH1 domains), a F(ab′)2 fragment (including bivalent fragments comprising two Fab fragments linked by a disulfide bridge at the hinge region), a Fd fragment (including fragments comprising the VH and CH1 fragment), a Fv fragment (including fragments comprising the VL and VH domains of a single arm of an antibody), a single-domain antibody (dAb or sdAb) (including fragments comprising a VH domain), an isolated complementarity determining region (CDR), a diabody (including fragments comprising bivalent dimers such as two VL and VH domains bound to each other and recognizing two different antigens), a fragment comprised of only a single monomeric variable domain, disulfide-linked Fvs (sdFv), an intrabody, an anti-idiotypic (anti-Id) antibody, or ab antigen-binding fragments thereof. In some instances, the libraries disclosed herein comprise nucleic acids encoding for an immunoglobulin, wherein the immunoglobulin is a Fv antibody, including Fv antibodies comprised of the minimum antibody fragment which contains a complete antigen-recognition and antigen-binding site. In some embodiments, the Fv antibody consists of a dimer of one heavy chain and one light chain variable domain in tight, non-covalent association, and the three hypervariable regions of each variable domain interact to define an antigen-binding site on the surface of the VH-VL dimer. In some embodiments, the six hypervariable regions confer antigen-binding specificity to the antibody. In some embodiments, a single variable domain (or half of an Fv comprising only three hypervariable regions specific for an antigen, including single domain antibodies isolated from camelid animals comprising one heavy chain variable domain such as VHH antibodies or nanobodies) has the ability to recognize and bind antigen. In some instances, the libraries disclosed herein comprise nucleic acids encoding for an immunoglobulin, wherein the immunoglobulin is a single-chain Fv or scFv, including antibody fragments comprising a VH, a VL, or both a VH and VL domain, wherein both domains are present in a single polypeptide chain. In some embodiments, the Fv polypeptide further comprises a polypeptide linker between the VH and VL domains allowing the scFv to form the desired structure for antigen binding. In some instances, a scFv is linked to the Fc fragment or a VHH is linked to the Fc fragment (including minibodies). In some instances, the antibody comprises immunoglobulin molecules and immunologically active fragments of immunoglobulin molecules, e.g., molecules that contain an antigen binding site. Immunoglobulin molecules are of any type (e.g., IgG, IgE, IgM, IgD, IgA and IgY), class (e.g., IgG 1, IgG 2, IgG 3, IgG 4, IgA 1 and IgA 2), or subclass.

In some embodiments, libraries comprise immunoglobulins that are adapted to the species of an intended therapeutic target. Generally, these methods include “mammalization” and comprise methods for transferring donor antigen-binding information to a less immunogenic mammal antibody acceptor to generate useful therapeutic treatments. In some instances, the mammal is mouse, rat, equine, sheep, cow, primate (e.g., chimpanzee, baboon, gorilla, orangutan, monkey), dog, cat, pig, donkey, rabbit, or human. In some instances, provided herein are libraries and methods for felinization and caninization of antibodies.

“Humanized” forms of non-human antibodies can be chimeric antibodies that contain minimal sequence derived from the non-human antibody. A humanized antibody is generally a human antibody (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. In some instances, these modifications are made to further refine antibody performance.

“Caninization” can comprise a method for transferring non-canine antigen-binding information from a donor antibody to a less immunogenic canine antibody acceptor to generate treatments useful as therapeutics in dogs. In some instances, caninized forms of non-canine antibodies provided herein are chimeric antibodies that contain minimal sequence derived from non-canine antibodies. In some instances, caninized antibodies are canine antibody sequences (“acceptor” or “recipient” antibody) in which hypervariable region residues of the recipient are replaced by hypervariable region residues from a non-canine species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the canine antibody are replaced by corresponding non-canine FR residues. In some instances, caninized antibodies include residues that are not found in the recipient antibody or in the donor antibody. In some instances, these modifications are made to further refine antibody performance. The caninized antibody may also comprise at least a portion of an immunoglobulin constant region (Fc) of a canine antibody.

“Felinization” can comprise a method for transferring non-feline antigen-binding information from a donor antibody to a less immunogenic feline antibody acceptor to generate treatments useful as therapeutics in cats. In some instances, felinized forms of non-feline antibodies provided herein are chimeric antibodies that contain minimal sequence derived from non-feline antibodies. In some instances, felinized antibodies are feline antibody sequences (“acceptor” or “recipient” antibody) in which hypervariable region residues of the recipient are replaced by hypervariable region residues from a non-feline species (“donor” antibody) such as mouse, rat, rabbit, cat, dogs, goat, chicken, bovine, horse, llama, camel, dromedaries, sharks, non-human primates, human, humanized, recombinant sequence, or an engineered sequence having the desired properties. In some instances, framework region (FR) residues of the feline antibody are replaced by corresponding non-feline FR residues. In some instances, felinized antibodies include residues that are not found in the recipient antibody or in the donor antibody. In some instances, these modifications are made to further refine antibody performance. The felinized antibody may also comprise at least a portion of an immunoglobulin constant region (Fc) of a felinize antibody.

Provided herein are libraries comprising nucleic acids encoding for a non-immunoglobulin. For example, the non-immunoglobulin is an antibody mimetic. Exemplary antibody mimetics include, but are not limited to, anticalins, affilins, affibody molecules, affimers, affitins, alphabodies, avimers, atrimers, DARPins, fynomers, Kunitz domain-based proteins, monobodies, anticalins, knottins, armadillo repeat protein-based proteins, and bicyclic peptides.

Libraries described herein comprising nucleic acids encoding for an immunoglobulin comprising variations in at least one region of the immunoglobulin. Exemplary regions of the antibody for variation include, but are not limited to, a complementarity-determining region (CDR), a variable domain, or a constant domain. In some instances, the CDR is CDR1, CDR2, or CDR3. In some instances, the CDR is a heavy domain including, but not limited to, CDRH1, CDRH2, and CDRH3. In some instances, the CDR is a light domain including, but not limited to, CDRL1, CDRL2, and CDRL3. In some instances, the variable domain is variable domain, light chain (VL) or variable domain, heavy chain (VH). In some instances, the VL domain comprises kappa or lambda chains. In some instances, the constant domain is constant domain, light chain (CL) or constant domain, heavy chain (CH).

Methods described herein provide for synthesis of libraries comprising nucleic acids encoding for an immunoglobulin, wherein each nucleic acid encodes for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the variant library comprises varied nucleic acids collectively encoding variations at multiple positions. In some instances, the variant library comprises sequences encoding for variation of at least a single codon of a CDRH1, CDRH2, CDRH3, CDRL1, CDRL2, CDRL3, VL, or VH domain. In some instances, the variant library comprises sequences encoding for variation of multiple codons of a CDRH1, CDRH2, CDRH3, CDRL1, CDRL2, CDRL3, VL, or VH domain. In some instances, the variant library comprises sequences encoding for variation of multiple codons of framework element 1 (FW1), framework element 2 (FW2), framework element 3 (FW3), or framework element 4 (FW4). An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

In some instances, the at least one region of the immunoglobulin for variation is from heavy chain V-gene family, heavy chain D-gene family, heavy chain J-gene family, light chain V-gene family, or light chain J-gene family. In some instances, the light chain V-gene family comprises immunoglobulin kappa (IGK) gene or immunoglobulin lambda (IGL). Exemplary genes include, but are not limited to, IGHV1-18, IGHV1-69, IGHV1-8, IGHV3-21, IGHV3-23, IGHV3-30/33rn, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGHV1-69, IGHV3-30, IGHV3-23, IGHV3, IGHV1-46, IGHV3-7, IGHV1, or IGHV1-8. In some instances, the gene is IGHV1-69 and IGHV3-30. In some instances, the gene is IGHJ3, IGHJ6, IGHJ, IGHJ4, IGHJS, IGHJ2, or IGH1. In some instances, the gene is IGHJ3, IGHJ6, IGHJ, or IGHJ4.

Provided herein are libraries comprising nucleic acids encoding for immunoglobulins, wherein the libraries are synthesized with various numbers of fragments. In some instances, the fragments comprise the CDRH1, CDRH2, CDRH3, CDRL1, CDRL2, CDRL3, VL, or VH domain. In some instances, the fragments comprise framework element 1 (FW1), framework element 2 (FW2), framework element 3 (FW3), or framework element 4 (FW4). In some instances, the immunoglobulin libraries are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

Libraries comprising nucleic acids encoding for immunoglobulins as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the immunoglobulins comprise at least or about 100, 200, 300, 400, 500, 600, 700, 800, 900, 1000, 2000, 3000, 4000, 5000, or more than 5000 amino acids.

A number of variant sequences for the at least one region of the immunoglobulin for variation are de novo synthesized using methods as described herein. In some instances, a number of variant sequences is de novo synthesized for CDRH1, CDRH2, CDRH3, CDRL1, CDRL2, CDRL3, VL, VH, or combinations thereof. In some instances, a number of variant sequences is de novo synthesized for framework element 1 (FW1), framework element 2 (FW2), framework element 3 (FW3), or framework element 4 (FW4). The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is at least or about 500, 600, 700, 800, 900, 1000, 2000, 3000, 4000, 5000, 6000, 7000, 8000, or more than 8000 sequences. In some instances, the number of variant sequences is about 10 to 500, 25 to 475, 50 to 450, 75 to 425, 100 to 400, 125 to 375, 150 to 350, 175 to 325, 200 to 300, 225 to 375, 250 to 350, or 275 to 325 sequences.

Variant sequences for the at least one region of the immunoglobulin, in some instances, vary in length or sequence. In some instances, the at least one region that is de novo synthesized is for CDRH1, CDRH2, CDRH3, CDRL1, CDRL2, CDRL3, VL, VH, or combinations thereof. In some instances, the at least one region that is de novo synthesized is for framework element 1 (FW1), framework element 2 (FW2), framework element 3 (FW3), or framework element 4 (FW4). In some instances, the variant sequence comprises at least or about 1, 2, 3, 4, 5, 6, 7, 8, 9, 10, 15, 20, 25, 30, 35, 40, 45, 50, or more than 50 variant nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 additional nucleotides or amino acids as compared to wild-type. In some instances, the variant sequence comprises at least or about 1, 2, 3, 4, 5, 6, 7, 8, 9, 10, 15, 20, 25, 30, 35, 40, 45, or 50 less nucleotides or amino acids as compared to wild-type. In some instances, the libraries comprise at least or about 10¹, 10², 10³, 10⁴, 10⁵, 10⁶, 10⁷, 10⁸, 10⁹, 10¹⁰, or more than 10¹⁰ variants.

Following synthesis of libraries described herein, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

In some instances, the libraries are assayed for functional activity, structural stability (e.g., thermal stable or pH stable), expression, specificity, or a combination thereof. In some instances, the libraries are assayed for immunoglobulin (e.g., an antibody) capable of folding. In some instances, a region of the antibody is assayed for functional activity, structural stability, expression, specificity, folding, or a combination thereof. For example, a VH region or VL region is assayed for functional activity, structural stability, expression, specificity, folding, or a combination thereof.

### DKK1 Libraries

Provided herein are DKK1 variant immunoglobulins (e.g., antibody, VHH) comprising nucleic acids encoding for immunoglobulins (e.g., antibodies) that bind to DKK1. In some instances, the immunoglobulin sequences for DKK1 binding domains are determined by interactions between the DKK1 binding domains and the DKK1.

Sequences of DKK1 binding domains based on surface interactions of DKK1 are analyzed using various methods. For example, multispecies computational analysis is performed. In some instances, a structure analysis is performed. In some instances, a sequence analysis is performed. Sequence analysis can be performed using a database known in the art. Non-limiting examples of databases include, but are not limited to, NCBI BLAST (blast.ncbi.nlm.nih.gov/Blast.cgi), UCSC Genome Browser (genome.ucsc.edu/), UniProt (www.uniprot.org/), and IUPHAR/BPS Guide to PHARMACOLOGY (guidetopharmacology.org/).

Described herein are DKK1 binding domains designed based on sequence analysis among various organisms. For example, sequence analysis is performed to identify homologous sequences in different organisms. Exemplary organisms include, but are not limited to, mouse, rat, equine, sheep, cow, primate (e.g., chimpanzee, baboon, gorilla, orangutan, monkey), dog, cat, pig, donkey, rabbit, fish, fly, and human.

Following identification of DKK1 binding domains, libraries comprising nucleic acids encoding for the DKK1 binding domains may be generated. In some instances, libraries of DKK1 binding domains comprise sequences of DKK1 binding domains designed based on conformational ligand interactions, peptide ligand interactions, small molecule ligand interactions, extracellular domains of DKK1, or antibodies that target DKK1. In some instances, libraries of DKK1 binding domains comprise sequences of DKK1 binding domains designed based on peptide ligand interactions. Libraries of DKK1 binding domains may be translated to generate protein libraries. In some instances, libraries of DKK1 binding domains are translated to generate peptide libraries, immunoglobulin libraries, derivatives thereof, or combinations thereof. In some instances, libraries of DKK1 binding domains are translated to generate protein libraries that are further modified to generate peptidomimetic libraries. In some instances, libraries of DKK1 binding domains are translated to generate protein libraries that are used to generate small molecules.

Methods described herein provide for synthesis of libraries of DKK1 binding domains comprising nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the libraries of DKK1 binding domains comprise varied nucleic acids collectively encoding variations at multiple positions. In some instances, the variant library comprises sequences encoding for variation of at least a single codon in a DKK1 binding domain. In some instances, the variant library comprises sequences encoding for variation of multiple codons in a DKK1 binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of libraries comprising nucleic acids encoding for the DKK1 binding domains, wherein the libraries comprise sequences encoding for variation of length of the DKK1 binding domains. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are DKK1 variant immunoglobulins (e.g., antibody, VHH) comprising nucleic acids encoding for immunoglobulins comprising DKK1 binding domains comprising variation in domain type, domain length, or residue variation. In some instances, the domain is a region in the immunoglobulin comprising the DKK1 binding domains. For example, the region is the VH, CDRH1, CDRH2, CDRH3, VL, CDRL1, CDRL2, or CDRL3 domain. In some instances, the domain is the DKK1 binding domain.

Methods described herein provide for synthesis of a DKK1 binding library of nucleic acids each encoding for a predetermined variant of at least one predetermined reference nucleic acid sequence. In some cases, the predetermined reference sequence is a nucleic acid sequence encoding for a protein, and the variant library comprises sequences encoding for variation of at least a single codon such that a plurality of different variants of a single residue in the subsequent protein encoded by the synthesized nucleic acid are generated by standard translation processes. In some instances, the DKK1 binding library comprises varied nucleic acids collectively encoding variations at multiple positions. In some instances, the variant library comprises sequences encoding for variation of at least a single codon of a VH, CDRH1, CDRH2, CDRH3, VL, CDRL1, CDRL2, or CDRL3 domain. In some instances, the variant library comprises sequences encoding for variation of at least a single codon in a DKK1 binding domain. In some instances, the variant library comprises sequences encoding for variation of multiple codons of a VH, CDRH1, CDRH2, CDRH3, VL, CDRL1, CDRL2, or CDRL3 domain. In some instances, the variant library comprises sequences encoding for variation of multiple codons in a DKK1 binding domain. An exemplary number of codons for variation include, but are not limited to, at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons.

Methods described herein provide for synthesis of a DKK1 binding library of nucleic acids each encoding for a predetermined variant of at least one predetermined reference nucleic acid sequence, wherein the DKK1 binding library comprises sequences encoding for variation of length of a domain. In some instances, the domain is VH, CDRH1, CDRH2, CDRH3, VL, CDRL1, CDRL2, or CDRL3 domain. In some instances, the domain is the DKK1 binding domain. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 225, 250, 275, 300, or more than 300 codons less as compared to a predetermined reference sequence. In some instances, the library comprises sequences encoding for variation of length of at least or about 1, 5, 10, 15, 20, 25, 30, 35, 40, 45, 50, 55, 60, 65, 70, 75, 80, 85, 90, 95, 100, 125, 150, 175, 200, 225, 250, 275, 300, or more than 300 codons more as compared to a predetermined reference sequence.

Provided herein are DKK1 variant immunoglobulins (e.g., antibody, VHH) comprising nucleic acids encoding for immunoglobulins comprising DKK1 binding domains, wherein the DKK1 binding libraries are synthesized with various numbers of fragments. In some instances, the fragments comprise the VH, CDRH1, CDRH2, CDRH3, VL, CDRL1, CDRL2, or CDRL3 domain. In some instances, the DKK1 variant immunoglobulins (e.g., antibody, VHH) are synthesized with at least or about 2 fragments, 3 fragments, 4 fragments, 5 fragments, or more than 5 fragments. The length of each of the nucleic acid fragments or average length of the nucleic acids synthesized may be at least or about 50, 75, 100, 125, 150, 175, 200, 225, 250, 275, 300, 325, 350, 375, 400, 425, 450, 475, 500, 525, 550, 575, 600, or more than 600 base pairs. In some instances, the length is about 50 to 600, 75 to 575, 100 to 550, 125 to 525, 150 to 500, 175 to 475, 200 to 450, 225 to 425, 250 to 400, 275 to 375, or 300 to 350 base pairs.

DKK1 variant immunoglobulins (e.g., antibody, VHH) comprising nucleic acids encoding for immunoglobulins comprising DKK1 binding domains as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 to about 75 amino acids.

DKK1 variant immunoglobulins (e.g., antibody, VHH) comprising de novo synthesized variant sequences encoding for immunoglobulins comprising DKK1 binding domains comprise a number of variant sequences. In some instances, a number of variant sequences is de novo synthesized for a CDRH1, CDRH2, CDRH3, CDRL1, CDRL2, CDRL3, VL, VH, or a combination thereof. In some instances, a number of variant sequences is de novo synthesized for framework element 1 (FW1), framework element 2 (FW2), framework element 3 (FW3), or framework element 4 (FW4). In some instances, a number of variant sequences is de novo synthesized for a GPCR binding domain. For example, the number of variant sequences is about 1 to about 10 sequences for the VH domain, about 10⁸ sequences for the DKK1 binding domain, and about 1 to about 44 sequences for the VL domain. The number of variant sequences may be at least or about 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, or more than 500 sequences. In some instances, the number of variant sequences is about 10 to 300, 25 to 275, 50 to 250, 75 to 225, 100 to 200, or 125 to 150 sequences.

Described herein are polypeptides that bind DKK1. In some embodiments, the polypeptides comprise a sequence as set forth in Table 2. In some embodiments, the polypeptide comprises an amino acid sequence of at least or about 70%, 80%, 85%, 90%, 91%, 92%, 93%, 94%, 95%, 96%, 97%, 98%, 99%, or 100% sequence identity to a sequence as set forth in Table 2. In some embodiments, the polypeptide comprises an amino acid sequence of at least or about 70%, 80%, 85%, 90%, 91%, 92%, 93%, 94%, 95%, 96%, 97%, 98%, 99%, or 100% identical to SEQ ID NO: 1, SEQ ID NO: 2, SEQ ID NO: 3, or SEQ ID NO: 4. In some embodiments, the polypeptide comprises an amino acid sequence of at least or about 70%, 80%, 85%, 90%, 91%, 92%, 93%, 94%, 95%, 96%, 97%, 98%, 99%, or 100% identical to SEQ ID NO: 20, SEQ ID NO: 21, SEQ ID NO: 22, SEQ ID NO: 23, or SEQ ID NO: 24.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “homology” or “similarity” between two proteins is determined by comparing the amino acid sequence and its conserved amino acid substitutes of one protein sequence to the second protein sequence. Similarity may be determined by procedures which are well-known in the art, for example, a BLAST program (Basic Local Alignment Search Tool at the National Center for Biological Information).

The terms “complementarity determining region,” and “CDR,” which are synonymous with “hypervariable region” or “HVR,” are known in the art to refer to non-contiguous sequences of amino acids within antibody variable regions, which confer antigen specificity and/or binding affinity. In general, there are three CDRs in each heavy chain variable region (CDRH1, CDRH2, CDRH3) and three CDRs in each light chain variable region (CDRL1, CDRL2, CDRL3). “Framework regions” and “FR” are known in the art to refer to the non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). The precise amino acid sequence boundaries of a given CDR or FR can be readily determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273, 927-948 (“Chothia” numbering scheme); MacCallum et al., J. Mol. Biol. 262:732-745 (1996), “Antibody-antigen interactions: Contact analysis and binding site topography,” J. Mol. Biol. 262, 732-745.” (“Contact” numbering scheme); Lefranc M P et al., “IMGT unique numbering for immunoglobulin and T cell receptor variable domains and Ig superfamily V-like domains,” Dev Comp Immunol, 2003 January; 27(1):55-77 (“IMGT” numbering scheme); Honegger A and Plückthun A, “Yet another numbering scheme for immunoglobulin variable domains: an automatic modeling and analysis tool,” J Mol Biol, 2001 Jun. 8; 309(3):657-70, (“Aho” numbering scheme); and Whitelegg N R and Rees A R, “WAM: an improved algorithm for modelling antibodies on the WEB,” Protein Eng. 2000 December; 13(12):819-24 (“AbM” numbering scheme. In certain embodiments the CDRs of the antibodies described herein can be defined by a method selected from Kabat, Chothia, IMGT, Aho, AbM, or combinations thereof.

The boundaries of a given CDR or FR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme.

DKK1 variant immunoglobulins (e.g., antibody, VHH) comprising de novo synthesized variant sequences encoding for immunoglobulins comprising DKK1 binding domains comprise improved diversity. For example, variants are generated by placing DKK1 binding domain variants in immunoglobulins comprising N-terminal CDRH3 variations and C-terminal CDRH3 variations. In some instances, variants include affinity maturation variants. Alternatively or in combination, variants include variants in other regions of the immunoglobulin including, but not limited to, CDRH1 and CDRH2. In some instances, the number of variants of the DKK1 variant immunoglobulins (e.g., antibody, VHH) is at least or about 10⁴, 10⁵, 10⁶, 10⁷, 10⁸, 10⁹, 10¹⁰, 10¹¹, 10¹², 10¹³, 10¹⁴, 10¹¹, 10¹⁶, 10¹⁷, 10¹⁸, 10¹⁹, 10²⁰, or more than 10²⁰ non-identical sequences.

In some instances, the at least one region of the antibody for variation is from heavy chain V-gene family, heavy chain D-gene family, heavy chain J-gene family, light chain V-gene family, or light chain J-gene family. In some instances, the light chain V-gene family comprises immunoglobulin kappa (IGK) gene or immunoglobulin lambda (IGL). Exemplary regions of the antibody for variation include, but are not limited to, IGHV1-18, IGHV1-69, IGHV1-8, IGHV3-21, IGHV3-23, IGHV3-30/33m, IGHV3-28, IGHV1-69, IGHV3-74, IGHV4-39, IGHV4-59/61, IGKV1-39, IGKV1-9, IGKV2-28, IGKV3-11, IGKV3-15, IGKV3-20, IGKV4-1, IGLV1-51, IGLV2-14, IGLV1-40, and IGLV3-1. In some instances, the gene is IGHV1-69, IGHV3-30, IGHV3-23, IGHV3, IGHV1-46, IGHV3-7, IGHV1, or IGHV1-8. In some instances, the gene is IGHV1-69 and IGHV3-30. In some instances, the region of the antibody for variation is IGHJ3, IGHJ6, IGHJ, IGHJ4, IGHJ5, IGHJ2, or IGH1. In some instances, the region of the antibody for variation is IGHJ3, IGHJ6, IGHJ, or IGHJ4. In some instances, the at least one region of the antibody for variation is IGHV1-69, IGHV3-23, IGKV3-20, IGKV1-39, or combinations thereof. In some instances, the at least one region of the antibody for variation is IGHV1-69 and IGKV3-20, In some instances, the at least one region of the antibody for variation is IGHV1-69 and IGKV1-39. In some instances, the at least one region of the antibody for variation is IGHV3-23 and IGKV3-20. In some instances, the at least one region of the antibody for variation is IGHV3-23 and IGKV1-39.

Provided herein are libraries comprising nucleic acids encoding for a DKK1 antibody comprising variation in at least one region of the antibody, wherein the at least one region is the CDR region. In some instances, the DKK1 antibody is a single domain antibody comprising one heavy chain variable domain such as a VHH antibody. In some instances, the VHH antibody comprises variation in one or more CDR regions. In some instances, libraries described herein comprise at least or about 1, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3. For example, the libraries comprise at least 2000 sequences of a CDR1, at least 1200 sequences for CDR2, and at least 1600 sequences for CDR3. In some instances, each sequence is non-identical.

In some instances, the CDR1, CDR2, or CDR3 is of a variable domain, light chain (VL). CDR1, CDR2, or CDR3 of a variable domain, light chain (VL) can be referred to as CDRL1, CDRL2, or CDRL3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VL. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VL. For example, the libraries comprise at least 20 sequences of a CDR1 of the VL, at least 4 sequences of a CDR2 of the VL, and at least 140 sequences of a CDR3 of the VL. In some instances, the libraries comprise at least 2 sequences of a CDR1 of the VL, at least 1 sequence of CDR2 of the VL, and at least 3000 sequences of a CDR3 of the VL. In some instances, the VL is IGKV1-39, IGKV1-9, IGKV2-28, IGKV3-11, IGKV3-15, IGKV3-20, IGKV4-1, IGLV1-51, IGLV2-14, IGLV1-40, or IGLV3-1. In some instances, the VL is IGKV2-28. In some instances, the VL is IGLV1-51.

In some instances, the CDR1, CDR2, or CDR3 is of a variable domain, heavy chain (VH). CDR1, CDR2, or CDR3 of a variable domain, heavy chain (VH) can be referred to as CDRH1, CDRH2, or CDRH3, respectively. In some instances, libraries described herein comprise at least or about 1, 2, 3, 4, 5, 6, 7, 8, 9, 10, 20, 30, 40, 50, 60, 70, 80, 90, 100, 200, 300, 400, 500, 600, 700, 800, 900, 1000, 1200, 1400, 1600, 1800, 2000, 2400, 2600, 2800, 3000, or more than 3000 sequences of a CDR1, CDR2, or CDR3 of the VH. In some instances, libraries described herein comprise at least or about 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences of a CDR1, CDR2, or CDR3 of the VH. For example, the libraries comprise at least 30 sequences of a CDR1 of the VH, at least 570 sequences of a CDR2 of the VH, and at least 10⁸ sequences of a CDR3 of the VH. In some instances, the libraries comprise at least 30 sequences of a CDR1 of the VH, at least 860 sequences of a CDR2 of the VH, and at least 10⁷ sequences of a CDR3 of the VH. In some instances, the VH is IGHV1-18, IGHV1-69, IGHV1-8 IGHV3-21, IGHV3-23, IGHV3-30/33m, IGHV3-28, IGHV3-74, IGHV4-39, or IGHV4-59/61. In some instances, the VH is IGHV1-69, IGHV3-30, IGHV3-23, IGHV3, IGHV1-46, IGHV3-7, IGHV1, or IGHV1-8. In some instances, the VH is IGHV1-69 or IGHV3-30. In some instances, the VH is IGHV3-23.

Libraries as described herein, in some embodiments, comprise varying lengths of a CDRL1, CDRL2, CDRL3, CDRH1, CDRH2, or CDRH3. In some instances, the length of the CDRL1, CDRL2, CDRL3, CDRH1, CDRH2, or CDRH3 comprises at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length. For example, the CDRH3 comprises at least or about 12, 15, 16, 17, 20, 21, or 23 amino acids in length. In some instances, the CDRL1, CDRL2, CDRL3, CDRH1, CDRH2, or CDRH3 comprises a range of about 1 to about 10, about 5 to about 15, about 10 to about 20, or about 15 to about 30 amino acids in length.

Libraries comprising nucleic acids encoding for antibodies having variant CDR sequences as described herein comprise various lengths of amino acids when translated. In some instances, the length of each of the amino acid fragments or average length of the amino acid synthesized may be at least or about 15, 20, 25, 30, 35, 40, 45, 50, 55, 60, 65, 70, 75, 80, 85, 90, 95, 100, 105, 110, 115, 120, 125, 130, 135, 140, 145, 150, or more than 150 amino acids. In some instances, the length of the amino acid is about 15 to 150, 20 to 145, 25 to 140, 30 to 135, 35 to 130, 40 to 125, 45 to 120, 50 to 115, 55 to 110, 60 to 110, 65 to 105, 70 to 100, or 75 to 95 amino acids. In some instances, the length of the amino acid is about 22 amino acids to about 75 amino acids. In some instances, the antibodies comprise at least or about 100, 200, 300, 400, 500, 600, 700, 800, 900, 1000, 2000, 3000, 4000, 5000, or more than 5000 amino acids.

Ratios of the lengths of a CDRL1, CDRL2, CDRL3, CDRH1, CDRH2, or CDRH3 may vary in libraries described herein. In some instances, a CDRL1, CDRL2, CDRL3, CDRH1, CDRH2, or CDRH3 comprising at least or about 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, or more than 90 amino acids in length comprises about 10%, 20%, 30%, 40%, 50%, 60%, 70%, 80%, 90%, or more than 90% of the library. For example, a CDRH3 comprising about 23 amino acids in length is present in the library at 40%, a CDRH3 comprising about 21 amino acids in length is present in the library at 30%, a CDRH3 comprising about 17 amino acids in length is present in the library at 20%, and a CDRH3 comprising about 12 amino acids in length is present in the library at 10%. In some instances, a CDRH3 comprising about 20 amino acids in length is present in the library at 40%, a CDRH3 comprising about 16 amino acids in length is present in the library at 30%, a CDRH3 comprising about 15 amino acids in length is present in the library at 20%, and a CDRH3 comprising about 12 amino acids in length is present in the library at 10%.

Libraries as described herein encoding for a VHH antibody comprise variant CDR sequences that are shuffled to generate a library with a theoretical diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences. In some instances, the library has a final library diversity of at least or about 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, or more than 10²⁰ sequences.

### DKK1 Binding Polypeptides

Provided herein polypeptides that bind DKK1. In some embodiments, the polypeptide that binds DKK1 comprises at least one VHH domain comprising a CDR1 comprising the amino acid sequence of SEQ ID NO: 5, a CDR2 comprising the amino acid sequence of SEQ ID NO: 6 or 29, and a CDR3 comprising the amino acid sequence of SEQ ID NO: 7 or 30. In some embodiments, at least one VHH domain is humanized. In some embodiments, a polypeptide that binds DKK1 comprises at least one VHH domain comprising the amino acid sequence of SEQ ID NO: 20.

In some embodiments, the polypeptide that binds DKK1 comprises at least one VHH domain comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10. In some embodiments, at least one VHH domain is humanized. In some embodiments, a polypeptide that binds DKK1 comprises at least one VHH domain comprising the amino acid sequence of SEQ ID NO: 21.

In some embodiments, the polypeptide that binds DKK1 comprises at least one VHH domain comprising a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13. In some embodiments, at least one VHH domain is humanized. In some embodiments, a polypeptide that binds DKK1 comprises at least one VHH domain comprising the amino acid sequence of SEQ ID NO: 22.

In some embodiments, the polypeptide that binds DKK1 comprises at least one VHH domain comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16. In some embodiments, at least one VHH domain is humanized. In some embodiments, a polypeptide that binds DKK1 comprises at least one VHH domain comprising the amino acid sequence of SEQ ID NO: 24.

In some embodiments, the polypeptide that binds DKK1 comprises at least one VHH domain comprising a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19. In some embodiments, at least one VHH domain is humanized. In some embodiments, a polypeptide that binds DKK1 comprises at least one VHH domain comprising the amino acid sequence of SEQ ID NO: 23.

In some embodiments, the polypeptide that binds DKK1 comprises at least two VHH domains, wherein a first VHH domain comprises a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a second VHH domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16. In some embodiments, at least one VHH domain is humanized. In some embodiments, a polypeptide that binds DKK1 comprises at least two VHH domains comprising the amino acid sequences of SEQ ID NO: 22 and 24.

In some embodiments, the polypeptide that binds DKK1 comprises at least two VHH domains, wherein a first VHH domain comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a second VHH domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16. In some embodiments, at least one VHH domain is humanized. In some embodiments, a polypeptide that binds DKK1 comprises at least two VHH domains comprising the amino acid sequences of SEQ ID NO: 23 and 24.

In some embodiments, a polypeptide that binds DKK1 comprises at least one VHH domain that binds DKK1 and an Fc region. In some embodiments, the Fc region is an antibody Fc region. In some embodiments, the Fc region is an IgG1, IgG2, IgG3, or IgG4. In some embodiments, the Fc region is an IgG2 (e.g., Table 2). In some embodiments, a polypeptide that binds DKK1 provided herein comprises two VHH domains that bind DKK1 and an Fc region. In some embodiments, the Fc region comprises a hinge that is capable of forming a dimer. In some embodiments, an Fc region mediates dimerization of the polypeptide that binds DKK1 at physiological conditions. In some embodiments, a dimer of polypeptides that bind DKK1 is formed comprising double the number of DKK1 binding sites. For example, a polypeptide that binds DKK1 comprising two VHH domains that bind DKK1 and an Fc region is divalent as a monomer, but at physiological conditions, the Fc region may mediate dimerization, such that the polypeptide that binds DKK1 is a tetravalent dimer under such conditions.

TABLE 2
Fc region amino acid sequence

| Fc region | SEQ | |
|---|---|---|
| description | ID | Sequence |
| IgG2 | 26 | VECPPCPAPPVAGPSVFLFPPKPKD |
| | | TLMISRTPEVTCVVVDVSHEDPEVQ |
| | | FNWYVDGVEVHNAKTKPREEQFNST |
| | | FRVVSVLTVVHQDWLNGKEYKCKVS |
| | | NKGLPAPIEKTISKTKGQPREPQVY |
| | | TLPPSREEMTKNQVSLTCLVKGFYP |
| | | SDIAVEWESNGQPENNYKTTPPMLD |
| | | SDGSFFLYSKLTVDKSRWQQGNVFS |
| | | CSVMHEALHNHYTQKSLSLSPG |

In some embodiments, the polypeptide that binds DKK1 comprises at least one VHH domain comprising a CDR1 comprising the amino acid sequence of SEQ ID NO: 5, a CDR2 comprising the amino acid sequence of SEQ ID NO: 6 or 29, and a CDR3 comprising the amino acid sequence of SEQ ID NO: 7 or 30, and an Fc region. In some embodiments, the Fc region comprises the amino acid sequence of SEQ ID NO:26. In some embodiments, at least one VHH domain is humanized.

In some embodiments, the polypeptide that binds DKK1 comprises at least one VHH domain comprising a CDR1 comprising the amino acid sequence of SEQ ID NO: 8, a CDR2 comprising the amino acid sequence of SEQ ID NO: 9, and a CDR3 comprising the amino acid sequence of SEQ ID NO: 10, and an Fc region. In some embodiments, the Fc region comprises the amino acid sequence of SEQ ID NO:26. In some embodiments, at least one VHH domain is humanized.

In some embodiments, the polypeptide that binds DKK1 comprises at least two VHH domains, wherein a first VHH domain comprises a CDR1 comprising the amino acid sequence of SEQ ID NO: 11, a CDR2 comprising the amino acid sequence of SEQ ID NO: 12, and a CDR3 comprising the amino acid sequence of SEQ ID NO: 13, and a second VHH domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, and an Fc region. In some embodiments, the Fc region comprises the amino acid sequence of SEQ ID NO:26. In some embodiments, at least one VHH domain is humanized. In some embodiments, a polypeptide that binds DKK1 comprises the amino acid sequence of SEQ ID NO: 3. In some embodiments, the polypeptide that binds DKK1 comprises the amino acid sequence of SEQ ID NO: 3 and can form a dimer under physiological conditions.

In some embodiments, the polypeptide that binds DKK1 comprises at least two VHH domains, wherein a first VHH domain comprises a CDR1 comprising the amino acid sequence of SEQ ID NO: 17, a CDR2 comprising the amino acid sequence of SEQ ID NO: 18, and a CDR3 comprising the amino acid sequence of SEQ ID NO: 19, and a second VHH domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16, and an Fc region. In some embodiments, the Fc region comprises the amino acid sequence of SEQ ID NO:26. In some embodiments, at least one VHH domain is humanized. In some embodiments, a polypeptide that binds DKK1 comprises the amino acid sequence of SEQ ID NO: 4. In some embodiments, the polypeptide that binds DKK1 comprises the amino acid sequence of SEQ ID NO: 4 and can form a dimer under physiological conditions.

In some embodiments, the polypeptide that binds DKK1 comprises a linker. In some embodiment, the linker is a flexible linker. In some embodiments, the linker is a rigid linker. In some embodiments, the linker is a charged linker. In some embodiments, the linker comprises glycine and/or serine residues. In some embodiments, the linker comprises glycine and serine residues (e.g., Table 3). In some embodiments, the linker further comprises alanine. In some embodiments, the linker is between about 1-50, 5-40, 10-30, or 20-25 amino acids in length. In some embodiments, the linker is about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acids in length. In some embodiments, the linker comprises the amino acid sequence of SEQ ID NO: 27 or 28. In some embodiments, the linker comprises the amino acid sequence of SEQ ID NO: 27. In some embodiments, the linker comprises the amino acid sequence of SEQ ID NO: 28.

TABLE 3
Linker amino acid sequences

| Linker | SEQ ID | Sequence |
|---|---|---|
| 1 | 27 | GGGGS |
| 2 | 28 | GGGGSGGGGSAS |

In some embodiments, from N-terminus to C-terminus, a polypeptide that binds DKK1 comprises the structure VHH-linker-Fc, wherein the VHH comprises the amino acid sequence of SEQ ID NO: 20, the linker comprises the amino acid sequence of SEQ ID NO: 27, and the Fc comprises the amino acid sequence of SEQ ID NO: 26, wherein the Fc region comprises a hinge. In some embodiments, the polypeptide that binds DKK1 comprises the amino acid sequence of SEQ ID NO: 1, which includes one VHH domain and an Fc region.

In some embodiments, from N-terminus to C-terminus, a polypeptide that binds DKK1 comprises the structure VHH-linker-Fc-linker-VHH, wherein the first VHH comprises the amino acid sequence of SEQ ID NO: 22, the first linker comprises the amino acid sequence of SEQ ID NO: 27, the Fc region comprises the amino acid sequence of SEQ ID NO: 26, wherein the Fc region comprises a hinge, the second linker comprises the amino acid sequence of SEQ ID NO: 28 and the second VHH comprises the amino acid sequence of SEQ ID NO: 24. In some embodiments, the polypeptide that binds DKK1 comprises the amino acid sequence of SEQ ID NO: 3, which includes two VHH domain and an Fc region.

In some embodiments, from N-terminus to C-terminus, a polypeptide that binds DKK1 comprises the structure VHH-linker-Fc-linker-VHH, wherein the first VHH comprises the amino acid sequence of SEQ ID NO: 23, the first linker comprises the amino acid sequence of SEQ ID NO: 27, the Fc region comprises the amino acid sequence of SEQ ID NO: 26, wherein the Fc region comprises a hinge, the second linker comprises the amino acid sequence of SEQ ID NO: 28 and the second VHH comprises the amino acid sequence of SEQ ID NO: 24. In some embodiments, the polypeptide that binds DKK1 comprises the amino acid sequence of SEQ ID NO: 4, which includes two VHH domain and an Fc region.

In some embodiments, a VHH domain that binds DKK1 may be humanized. Humanized antibodies (such as sdAbs or VHH-containing polypeptides) are useful as therapeutic molecules because humanized antibodies reduce or eliminate the human immune response to non-human antibodies, which can result in an immune response to an antibody therapeutic, and decreased effectiveness of the therapeutic. Generally, a humanized antibody comprises one or more variable domains in which CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (for example, the antibody from which the CDR residues are derived), for example, to restore or improve antibody specificity or affinity.

Humanized antibodies and methods of making them are reviewed, for example, in Almagro and Fransson, (2008) Front. Biosci. 13: 1619-1633, and are further described, for example, in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34; Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J. Cancer, 83:252-260 (describing the “guided selection” approach to FR shuffling).

Human framework regions that can be used for humanization include but are not limited to: framework regions selected using the “best-fit” method (see, for example, Sims et al. (1993) J. Immunol. 151:2296); framework regions derived from the consensus sequence of human antibodies of a particular subgroup of heavy chain variable regions (see, for example, Carter et al. (1992) Proc. Natl. Acad. Sci. USA, 89:4285; and Presta et al. (1993) J. Immunol, 151:2623); human mature (somatically mutated) framework regions or human germline framework regions (see, for example, Almagro and Fransson, (2008) Front. Biosci. 13:1619-1633); and framework regions derived from screening FR libraries (see, for example, Baca et al., (1997) J. Biol. Chem. 272: 10678-10684 and Rosok et al., (1996) J. Biol. Chem. 271:22611-22618). Typically, the FR regions of a VHH are replaced with human FR regions to make a humanized VHH. In some embodiments, certain FR residues of the human FR are replaced in order to improve one or more properties of the humanized VHH. VHH domains with such replaced residues are still referred to herein as “humanized.”

In various embodiments, an Fc region included in a polypeptide that binds DKK1 is a human Fc region, or is derived from a human Fc region. In some embodiments, the Fc region is a human IgG. In some embodiments, the Fc region is a human IgG1, IgG2, IgG3, or IgG4. In some embodiments, the Fc region is a human IgG2.

In some embodiments, the polypeptide that binds DKK1 comprises: a) a first antibody variable domain comprising a CDR1 comprising the amino acid of SEQ ID NO: 11 or 17, a CDR2 comprising the amino acid sequence of SEQ ID NO: 12 or 18, and a CDR3 comprising the amino acid sequence of SEQ ID NO: 13 or 19; and b) a second antibody variable domain comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, the polypeptide that binds DKK1 comprises (ii) a first antibody variable domain comprising a CDR1 comprising the amino acid of SEQ ID NO: 11, a CDR2 comprising the amino acid sequence of SEQ ID NO: 12, and a CDR3 comprising the amino acid sequence of SEQ ID NO: 13; and (ii) a second antibody variable domain comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

In some embodiments, the polypeptide that binds to DKK1 comprises a CDR comprising the amino acid sequence of any one of SEQ ID NOs: 5-19 and 29-30, or a functional variant thereof having one or two amino acid substitutions with respect to the amino acid sequence of any one of SEQ ID NOs; 5-19 and 29-30. A “functional variant” as described herein with respect to a complementarity determining region (CDR) refers to a variant of a CDR having one or more amino acid modification(s) (e.g., amino acid insertions, substitutions, or deletions) with respect to a CDR sequence of a variable domain that binds to a particular antigen, where the one or more amino acid modification(s) to the CDR does not result in ablation of antigen binding of the variable domain to the particular antigen.

In some embodiments, the polypeptide that binds DKK1 comprises a first antibody variable domain comprising an amino acid sequence at least 80%, at least 85%, at least 90%, at least 95%, at least 96%, at least 97%, at least 98%, or at least 99% identical to the amino acid sequence of SEQ ID NO: 22 or SEQ ID NO: 23.

In some embodiments, the polypeptide that binds DKK1 comprises a second antibody variable domain comprising an amino acid sequence at least 80%, at least 85%, at least 90%, at least 95%, at least 96%, at least 97%, at least 98%, or at least 99% identical to the amino acid sequence of the amino acid sequence of SEQ ID NO: 24.

In some embodiments, the polypeptide that binds DKK1 comprises (i) a first antibody variable domain comprising an amino acid sequence at least 80%, at least 85%, at least 90%, at least 95%, at least 96%, at least 97%, at least 98%, or at least 99% identical to the amino acid sequence of SEQ ID NO: 22 or SEQ ID NO: 23 and (ii) a second antibody variable domain comprising an amino acid sequence at least 80%, at least 85%, at least 90%, at least 95%, at least 96%, at least 97%, at least 98%, or at least 99% identical to the amino acid sequence of the amino acid sequence of SEQ ID NO: 24.

In some embodiments, the polypeptide that binds DKK1 comprises a first antibody variable domain comprising the amino acid sequence of SEQ ID NO: 22 or SEQ ID NO: 23. In some embodiments, the polypeptide that binds DKK1 comprises a second antibody variable domain comprising the amino acid sequence of SEQ ID NO: 24. In some embodiments, the polypeptide that binds DKK1 comprises (i) a first antibody variable domain comprising the amino acid sequence of SEQ ID NO: 22 or SEQ ID NO: 23 and (ii) the polypeptide that binds DKK1 comprises a second antibody variable domain comprising the amino acid sequence of SEQ ID NO: 24.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 3 or SEQ ID NO: 4. In some embodiments, the polypeptide that binds DKK1 comprises the amino acid sequence of SEQ ID NO: 3 or SEQ ID NO: 4. In some embodiments, the polypeptide that binds DKK1 comprises the amino acid sequence of SEQ ID NO: 3. In some embodiments, the polypeptide that binds DKK1 comprises the amino acid sequence of SEQ ID NO: 4.

In some embodiments, the polypeptide that binds DKK1 comprises an antibody variable domain comprising a CDR1 comprising the amino acid of SEQ ID NO: 5, a CDR2 comprising the amino acid sequence of SEQ ID NO: 6 or 29, and a CDR3 comprising the amino acid sequence of SEQ ID NO: 7 or 30.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 20. In some embodiments, the polypeptide that binds DKK1 comprises the amino acid sequence of SEQ ID NO: 20.

In some embodiments, the polypeptide that binds DKK1 comprises an Fc region. In some embodiments, the Fc region is an IgG1, IgG2, IgG3, or IgG4 Fc region. In some embodiments, the polypeptide that binds DKK1 has the structure: first antibody variable domain-linker-Fc region-linker-second antibody variable domain.

In some embodiments, the Fc region having one or more amino acid modification(s) with respect to a wildtype Fc region that improves the serum half-life of the antibody or antigen-binding fragment thereof. For example, an antibody or antigen-binding fragment thereof can comprise a modified IgG Fc region for improved serum half-life having one or more of the following modifications with respect to a wildtype IgG Fc region: M252Y, S254T, T256E, M428L, N434S, T256D, T307R, Q311V, N315D, N286D, T307R, H285N, or T307Q. In some embodiments, an antibody or antigen-binding fragment thereof can comprise a modified IgG Fc region for improved serum half-life having one of the following sets of modifications: M252Y/S254T/T256E, M428L/N434S, T256D/T307R/Q311V, T256D/N315D/A378V, T256D/N286D/T307R/Q311V, H285N/T307Q/N315D, T256D/T307R/Q311V/A378V, H285D/Q311V/A378V, T256D/H285D/A378V, T256D/Q311V/A378V, T256D/H285D/N286D/T307R/A378V, T256D/H286D/T307R/Q311V/A378V, T307Q/Q311V/A378V, H285D/T307Q/A378V, or T256D/H285D/T307R/Q311V/A378V. In some embodiments, an antibody or antigen-binding fragment thereof can comprise an Fc region having one or more deletions with respect to a wildtype Fc region for improved serum half-life. For example, an Fc region can comprise a deletion of a CH2 domain, a CH3 domain, or a portion thereof for improved serum half-life. In some embodiments, an Fc region can comprise a deletion of a C-terminal amino acid in a CH3 domain, such as a C-terminal lysine, for improved serum half-life.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 1. In some embodiments the polypeptide comprises the amino acid sequence of SEQ ID NO: 1. In some embodiments, the one or more antibody variable domain is a VHH domain.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 2. In some embodiments the polypeptide comprises the amino acid sequence of SEQ ID NO: 2. In some embodiments, the one or more antibody variable domain is a VHH domain.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 3. In some embodiments the polypeptide comprises the amino acid sequence of SEQ ID NO: 3. In some embodiments, the one or more antibody variable domain is a VHH domain.

In some embodiments, the polypeptide that binds DKK1 comprises an amino acid sequence at least 80%, at least 85%, at least 90%, at least 95%, at least 96%, at least 97%, at least 98%, or at least 99% identical to the amino acid sequence of SEQ ID NO: 4. In some embodiments the polypeptide comprises the amino acid sequence of SEQ ID NO: 4. In some embodiments, the one or more antibody variable domain is a VHH domain.

Provided herein are polypeptides that bind DKK1 (e.g., antibody, VHH). In some instances, the polypeptide is an antibody. In some instances, the polypeptide is a VHH antibody. In some instances, the polypeptide comprises a binding affinity (e.g., kD) to DKK1 of less than 1 nM, less than 1.2 nM, less than 2 nM, less than 5 nM, less than 10 nM, less than 11 nm, less than 13.5 nM, less than 15 nM, less than 20 nM, less than 25 nM, or less than 30 nM. In some instances, the polypeptide binds to DKK1 with a kD of less than 1 nM. In some instances, the polypeptide binds to DKK1 with a kD of less than 1.2 nM. In some instances, the polypeptide binds to DKK1 with a kD of less than 2 nM. In some instances, the polypeptide binds to DKK1 with a kD of less than 5 nM. In some instances, the polypeptide binds to DKK1 with a kD of less than 10 nM. In some instances, the polypeptide binds to DKK1 with a kD of less than 13.5 nM. In some instances, the polypeptide binds to DKK1 with a kD of less than 15 nM. In some instances, the polypeptide binds to DKK1 with a kD of less than 20 nM. In some instances, the polypeptide binds to DKK1 with a kD of less than 25 nM. In some instances, the polypeptide binds to DKK1 with a kD of less than 30 nM.

Provided herein are polypeptides that bind DKK1 (e.g., antibody, VHH) and have a long half-life. In some instances, the half-life of the polypeptide that binds DKK1 is at least or about 12 hours, 24 hours 36 hours, 48 hours, 60 hours, 72 hours, 84 hours, 96 hours, 108 hours, 120 hours, 140 hours, 160 hours, 180 hours, 200 hours, or more than 200 hours. In some instances, the half-life of the polypeptide that binds DKK1 is in a range of about 12 hours to about 300 hours, about 20 hours to about 280 hours, about 40 hours to about 240 hours, or about 60 hours to about 200 hours.

Polypeptides that bind DKK1 as described herein may comprise improved properties. In some instances, the polypeptides that bind DKK1 are monomeric. In some instances, the polypeptides that bind DKK1 are not prone to aggregation. In some instances, at least or about 70%, 75%, 80%, 85%, 90%, 95%, or 99% of the polypeptides that bind DKK1 are monomeric. In some instances, the polypeptides that bind DKK1 are thermostable. In some instances, the polypeptides that bind DKK1 result in reduced non-specific binding.

Following synthesis of DKK1 variant immunoglobulins (e.g., antibody, VHH) comprising nucleic acids encoding immunoglobulins comprising DKK1 binding domains, libraries may be used for screening and analysis. For example, libraries are assayed for library displayability and panning. In some instances, displayability is assayed using a selectable tag. Exemplary tags include, but are not limited to, a radioactive label, a fluorescent label, an enzyme, a chemiluminescent tag, a colorimetric tag, an affinity tag or other labels or tags that are known in the art. In some instances, the tag is histidine, polyhistidine, myc, hemagglutinin (HA), or FLAG. In some instances, the DKK1 variant immunoglobulins (e.g., antibody, VHH) comprises nucleic acids encoding immunoglobulins with multiple tags such as GFP, FLAG, and Lucy as well as a DNA barcode. In some instances, libraries are assayed by sequencing using various methods including, but not limited to, single-molecule real-time (SMRT) sequencing, Polony sequencing, sequencing by ligation, reversible terminator sequencing, proton detection sequencing, ion semiconductor sequencing, nanopore sequencing, electronic sequencing, pyrosequencing, Maxam-Gilbert sequencing, chain termination (e.g., Sanger) sequencing, +S sequencing, or sequencing by synthesis.

### Polypeptide Expression and Production

Nucleic acid molecules comprising polynucleotides that encode a polypeptide that binds DKK1 are provided. In some embodiments, the nucleic acid molecule may also encode a leader sequence that directs secretion of the polypeptide that binds DKK1, which leader sequence is typically cleaved such that it is not present in the secreted polypeptide. The leader sequence may be a native heavy chain (or VHH) leader sequence, or may be another heterologous leader sequence.

Nucleic acid molecules can be constructed using recombinant DNA techniques conventional in the art. In some embodiments, a nucleic acid molecule is an expression vector that is suitable for expression in a selected host cell.

Vectors comprising nucleic acids that encode the polypeptide that binds DKK1 described herein are provided. Such vectors include, but are not limited to, DNA vectors, phage vectors, viral vectors, retroviral vectors, etc. In some embodiments, a vector is selected that is optimized for expression of polypeptides in a desired cell type, such as CHO or CHO-derived cells, or in NSO cells. Exemplary such vectors are described, for example, in Running Deer et al., Biotechnol. Prog. 20:880-889 (2004).

In some embodiments, a polypeptide that binds DKK1 may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells (Crucell); and NSO cells. In some embodiments, the polypeptide that binds DKK1 may be expressed in yeast. See, e.g., U.S. Publication No. US 2006/0270045 A1. In some embodiments, a particular eukaryotic host cell is selected based on its ability to make desired post-translational modifications to the polypeptide. For example, in some embodiments, CHO cells produce polypeptides that have a higher level of sialylation than the same polypeptide produced in 293 cells.

Introduction of one or more nucleic acids (such as vectors) into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, for example, in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Host cells comprising any of the nucleic acids or vectors described herein are also provided. In some embodiments, a host cell that expresses a polypeptide that binds DKK1 described herein is provided. The DKK1-binding polypeptides expressed in host cells can be purified by any suitable method. Such methods include, but are not limited to, the use of affinity matrices or hydrophobic interaction chromatography. Suitable affinity ligands include the ROR1 ECD and agents that bind Fc regions. For example, a Protein A, Protein G, Protein A/G, or an antibody affinity column may be used to bind the Fc region and to purify the polypeptide that binds DKK1 that comprises an Fc region. Hydrophobic interactive chromatography, for example, a butyl or phenyl column, may also be suitable for purifying some polypeptides such as antibodies. Ion exchange chromatography (for example anion exchange chromatography and/or cation exchange chromatography) may also be suitable for purifying some polypeptides such as antibodies. Mixed-mode chromatography (for example reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art.

In some embodiments, the polypeptide that binds DKK1 is produced in a cell-free system. Nonlimiting exemplary cell-free systems are described, for example, in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some embodiments, a polypeptide that binds DKK1 is prepared by the methods described above are provided. In some embodiments, the polypeptide that binds DKK1 is prepared in a host cell. In some embodiments, the polypeptide that binds DKK1 is prepared in a cell-free system. In some embodiments, the polypeptide that binds DKK1 is purified.

In some embodiments, compositions comprising polypeptides that bind DKK!prepared by the methods described above are provided. In some embodiments, the composition comprises a polypeptide that binds DKK1 prepared in a host cell. In some embodiments, the composition comprises a polypeptide that binds DKK1 prepared in a cell-free system. In some embodiments, the composition comprises a purified polypeptide that binds DKK1.

### Pharmaceutical Compositions

Pharmaceutical compositions comprising a polypeptide that binds DKK1 are provided. In some embodiments, the polypeptide that binds DKK1 is any one of the polypeptides described herein. In some embodiments, the pharmaceutical composition further comprises a pharmaceutically acceptable carrier.

Accordingly, described herein are pharmaceutical compositions comprising polypeptides that bind DKK1. In some embodiments, the polypeptide that binds DKK1 comprises the amino acid sequence of SEQ ID NO: 1, 2, 3, 4, 20, 21, 22, 23, or 24. In some embodiments, the polypeptide that binds DKK1 comprises an amino acid sequence that is at least or about 70%, 80%, 85%, 90%, 91%, 92%, 93%, 94%, 95%, 96%, 97%, 98%, 99%, or 100% identical to SEQ ID NO: 1, 2, 3, 4, 20, 21, 22, 23, or 24.

In some instances, a pharmaceutical composition comprises a polypeptide that binds DKK1 described herein comprising an amino acid sequence of SEQ ID NO: 1, SEQ ID NO: 2, SEQ ID NO: 3, SEQ ID NO: 4, SEQ ID NO: 20, SEQ ID NO: 21, SEQ ID NO: 22, SEQ ID NO: 23, or SEQ ID NO: 24. In some instances, a pharmaceutical composition comprises a polypeptide that binds DKK1 described herein comprising an amino acid sequence that is at least 80% identical to the sequence of SEQ ID NO: 1, 2, 3, 4, 20, 21, 22, 23, or 24. In some instances, a pharmaceutical composition comprises a polypeptide that binds DKK1 described herein comprising an amino acid sequence that is at least 85% identical to SEQ ID NO: 1, 2, 3, 4, 20, 21, 22, 23, or 24. In some instances, a pharmaceutical composition comprises a polypeptide that binds DKK1 described herein comprising an amino acid sequence that is at least 90% identical to SEQ ID NO: 1, 2, 3, 4, 20, 21, 22, 23, or 24. In some instances, a pharmaceutical composition comprises a polypeptide that binds DKK1 described herein comprising an amino acid sequence that is at least 95% identical to SEQ ID NO: 1, 2, 3, 4, 20, 21, 22, 23 or 24.

### Methods of Treatment

Provided herein are polypeptides that bind DKK1 (e.g., antibody, VHH) that may have therapeutic effects. In some instances, the polypeptides that bind DKK1 variant immunoglobulins (e.g., antibody, VHH) may be used to treat a disease or disorder.

Exemplary diseases include, but are not limited to, DKK1 cancer (e.g., gastro-esophageal cancer, endometrial cancer, ovarian cancer, prostate cancer, liver cancer, etc.), inflammatory diseases or disorders, a metabolic disease or disorder, a cardiovascular disease or disorder, a respiratory disease or disorder, pain, a digestive disease or disorder, a reproductive disease or disorder, an endocrine disease or disorder, or a neurological disease or disorder. In some instances, the cancer is a solid cancer or a hematologic cancer. In some instances, a modulator of DKK1 as described herein is used for treatment of weight gain (or for inducing weight loss), treatment of obesity, or treatment of Type II diabetes. In some instances, the DKK1 modulator is used for treating hypoglycemia. In some instances, the DKK1 modulator is used for treating post-bariatric hypoglycemia. In some instances, the DKK1 modulator is used for treating severe hypoglycemia. In some instances, the DKK1 modulator is used for treating hyperinsulinism. In some instances, the DKK1 modulator is used for treating congenital hyperinsulinism.

DKK1 can be tumorigenic in cancer. DKK1 can also be immunosuppressive (e.g., via myeloid-derived suppressor cells (MDSCs) or natural killer (NK) cells). DKK1 can lead to immune suppression through T cell inactivation, MDSC accumulation, or NK cell clearance. DKK1 can inhibit Wnt binding to low-density lipoprotein (LDL) receptor related protein 5 (LRP5). DKK1 can inhibit Wnt binding to LDL receptor related protein 6 (LRP6). DKK1 can inhibit Wnt binding to an LRP5/6 complex. Mutations in Wnt activating genes can lead to increased DKK1 expression. In some embodiments, the polypeptides that bind DKK1 may be used to treat a subject having a cancer with an elevated expression of DKK1.

Antagonist mAb can activate an innate immune response with anti-angiogenic and direct antitumor effects, binding and removing DKK1 from the tumor microenvironment. Tumors with Wnt activating mutations can responded to DKK1 antagonism. For example, high tumoral DKK1 can be associated with longer progression-free survival in esophagogastric cancer patients.

Provided herein are methods of treating disease in an individual comprising administering a polypeptide that binds DKK1 or a pharmaceutical composition thereof. In some embodiments, methods for treating cancer in an individual are provided.

In some embodiments, the method comprises administering to the individual a pharmaceutically effective amount of a polypeptide that binds DKK1 as provided herein. Such methods of treatment may be in humans or animals. In some embodiments, methods of treating humans are provided. Nonlimiting exemplary cancers that may be treated with polypeptide that binds DKK1 provided herein include non-small cell lung cancer, multiple myeloma, hepatocellular carcinoma, esophageal cancer, gastric cancer, esophagogastric cancer, biliary tract cancer, pancreatic cancer, gastric cancer, cholangiocarcinoma, laryngeal squamous cell carcinoma, hepatocellular carcinoma, endometrial cancer, cervical cancer, ovarian cancer, liver cancer, prostate cancer, and breast cancer.

The polypeptide that binds DKK1 can be administered as needed to subjects. Determination of the frequency of administration can be made by persons skilled in the art, such as an attending physician based on considerations of the condition being treated, age of the subject being treated, severity of the condition being treated, general state of health of the subject being treated and the like.

In some embodiments, pharmaceutical formulations are administered in a pharmaceutically effective amount for treating (including prophylaxis of) cancer.

In some instances, the subject is a mammal. In some instances, the subject is a human. Subjects treated by methods described herein may be infants, adults, or children.

In some embodiments, DKK1-binding polypeptides can be administered in vivo by various routes, including, but not limited to, intramuscular, intravenous, intra-arterial, parenteral, intraperitoneal or subcutaneous. The appropriate formulation and route of administration may be selected according to the intended application.

### Kits

Also provided are articles of manufacture and kits that include any of the polypeptides that bind to DKK1 provided herein and suitable packaging. In some embodiments, the invention includes a kit with (i) a polypeptide that binds DKK1, and (ii) instructions for using the kit to administer the polypeptide to an individual. In some embodiments, the invention includes a kit with (i) a pharmaceutical composition comprising a polypeptide that binds DKK1, and (ii) instructions for using the kit to administer the pharmaceutical composition to an individual.

Suitable packaging for compositions described herein are known in the art, and include, for example, vials (e.g., sealed vials), vessels, ampules, bottles, jars, flexible packaging (e.g., sealed Mylar or plastic bags), and the like. These articles of manufacture may further be sterilized and/or sealed. Instructions supplied in the kits of the invention are typically written instructions on a label or package insert (e.g., a paper sheet included in the kit), but machine-readable instructions (e.g., instructions carried on a magnetic or optical storage disk) are also acceptable. The kit may further comprise a description of selecting an individual suitable or treatment.

### Expression Systems

Provided herein are libraries comprising nucleic acids encoding for immunoglobulins comprising DKK1 binding domains, wherein the libraries have improved specificity, stability, expression, folding, or downstream activity. In some instances, libraries described herein are used for screening and analysis.

Provided herein are libraries comprising nucleic acids encoding for immunoglobulins comprising DKK1 binding domains, wherein the nucleic acid libraries are used for screening and analysis. In some instances, screening and analysis comprise in vitro, in vivo, or ex vivo assays. Cells for screening include primary cells taken from living subjects or cell lines. Cells may be from prokaryotes (e.g., bacteria and fungi) or eukaryotes (e.g., animals and plants). Exemplary animal cells include, without limitation, those from a mouse, rabbit, primate, and insect. In some instances, cells for screening include a cell line including, but not limited to, Chinese Hamster Ovary (CHO) cell line, human embryonic kidney (HEK) cell line, or baby hamster kidney (BHK) cell line. In some instances, nucleic acid libraries described herein may also be delivered to a multicellular organism. Exemplary multicellular organisms include, without limitation, a plant, a mouse, rabbit, primate, and insect.

Nucleic acid libraries or protein libraries encoded thereof described herein may be screened for various pharmacological or pharmacokinetic properties. In some instances, the libraries are screened using in vitro assays, in vivo assays, or ex vivo assays. For example, in vitro pharmacological or pharmacokinetic properties that are screened include, but are not limited to, binding affinity, binding specificity, and binding avidity. Exemplary in vivo pharmacological or pharmacokinetic properties of libraries described herein that are screened include, but are not limited to, therapeutic efficacy, activity, preclinical toxicity properties, clinical efficacy properties, clinical toxicity properties, immunogenicity, potency, and clinical safety properties.

Pharmacological or pharmacokinetic properties that may be screened include, but are not limited to, cell binding affinity and cell activity. For example, cell binding affinity assays or cell activity assays are performed to determine agonistic, antagonistic, or allosteric effects of libraries described herein. In some instances, libraries as described herein are compared to cell binding or cell activity of ligands of DKK1.

Libraries as described herein may be screened in cell-based assays or in non-cell-based assays. Examples of non-cell-based assays include, but are not limited to, using viral particles, using in vitro translation proteins, and using proteoliposomes with DKK1.

Nucleic acid libraries as described herein may be screened by sequencing. In some instances, next generation sequence is used to determine sequence enrichment of DKK1 binding variants. In some instances, V gene distribution, J gene distribution, V gene family, CDR3 counts per length, or a combination thereof is determined. In some instances, clonal frequency, clonal accumulation, lineage accumulation, or a combination thereof is determined. In some instances, number of sequences, sequences with VH clones, clones, clones greater than 1, clonotypes, clonotypes greater than 1, lineages, simpsons, or a combination thereof is determined. In some instances, a percentage of non-identical CDR3s is determined. For example, the percentage of non-identical CDR3s is calculated as the number of non-identical CDR3s in a sample divided by the total number of sequences that had a CDR3 in the sample.

Provided herein are nucleic acid libraries and polypeptides, wherein the nucleic acid libraries and polypeptides may be expressed in a vector. Expression vectors for inserting nucleic acids encoding the polypeptides disclosed herein may comprise eukaryotic or prokaryotic expression vectors. Exemplary expression vectors include, without limitation, mammalian expression vectors: pSF-CMV-NEO-NH2-PPT-3XFLAG, pSF-CMV-NEO—COOH-3XFLAG, pSF-CMV—PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEFla-mCherry-N1 Vector, pEFla-tdTomato Vector, pSF-CMV-FMDV-Hygro, pSF-CMV-PGK-Puro, pMCP-tag(m), and pSF-CMV—PURO-NH2-CMYC; bacterial expression vectors: pSF-OXB20-BetaGal,pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8. In some instances, the vector is pcDNA3 or pcDNA3.1.

Described herein are nucleic acid libraries that are expressed in a vector to generate a construct comprising an immunoglobulin comprising sequences of DKK1 binding domains. In some instances, a size of the construct varies. In some instances, the construct comprises at least or about 500, 600, 700, 800, 900, 1000, 1100, 1300, 1400, 1500, 1600, 1700, 1800, 2000, 2400, 2600, 2800, 3000, 3200, 3400, 3600, 3800, 4000, 4200, 4400, 4600, 4800, 5000, 6000, 7000, 8000, 9000, 10000, or more than 10000 bases. In some instances, a the construct comprises a range of about 300 to 1,000, 300 to 2,000, 300 to 3,000, 300 to 4,000, 300 to 5,000, 300 to 6,000, 300 to 7,000, 300 to 8,000, 300 to 9,000, 300 to 10,000, 1,000 to 2,000, 1,000 to 3,000, 1,000 to 4,000, 1,000 to 5,000, 1,000 to 6,000, 1,000 to 7,000, 1,000 to 8,000, 1,000 to 9,000, 1,000 to 10,000, 2,000 to 3,000, 2,000 to 4,000, 2,000 to 5,000, 2,000 to 6,000, 2,000 to 7,000, 2,000 to 8,000, 2,000 to 9,000, 2,000 to 10,000, 3,000 to 4,000, 3,000 to 5,000, 3,000 to 6,000, 3,000 to 7,000, 3,000 to 8,000, 3,000 to 9,000, 3,000 to 10,000, 4,000 to 5,000, 4,000 to 6,000, 4,000 to 7,000, 4,000 to 8,000, 4,000 to 9,000, 4,000 to 10,000, 5,000 to 6,000, 5,000 to 7,000, 5,000 to 8,000, 5,000 to 9,000, 5,000 to 10,000, 6,000 to 7,000, 6,000 to 8,000, 6,000 to 9,000, 6,000 to 10,000, 7,000 to 8,000, 7,000 to 9,000, 7,000 to 10,000, 8,000 to 9,000, 8,000 to 10,000, or 9,000 to 10,000 bases.

Provided herein are libraries comprising nucleic acids encoding for immunoglobulins, wherein the nucleic acid libraries are expressed in a cell. In some instances, the libraries are synthesized to express a reporter gene. Exemplary reporter genes include, but are not limited to, acetohydroxyacid synthase (AHAS), alkaline phosphatase (AP), beta galactosidase (LacZ), beta glucoronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination.

### Variant Libraries

### Codon Variation

Variant nucleic acid libraries described herein may comprise a plurality of nucleic acids, wherein each nucleic acid encodes for a variant codon sequence compared to a reference nucleic acid sequence. In some instances, each nucleic acid of a first nucleic acid population contains a variant at a single variant site. In some instances, the first nucleic acid population contains a plurality of variants at a single variant site such that the first nucleic acid population contains more than one variant at the same variant site. The first nucleic acid population may comprise nucleic acids collectively encoding multiple codon variants at the same variant site. The first nucleic acid population may comprise nucleic acids collectively encoding up to 19 or more codons at the same position. The first nucleic acid population may comprise nucleic acids collectively encoding up to 60 variant triplets at the same position, or the first nucleic acid population may comprise nucleic acids collectively encoding up to 61 different triplets of codons at the same position. Each variant may encode for a codon that results in a different amino acid during translation.

A nucleic acid population may comprise varied nucleic acids collectively encoding up to 20 codon variations at multiple positions. In such cases, each nucleic acid in the population comprises variation for codons at more than one position in the same nucleic acid. In some instances, each nucleic acid in the population comprises variation for codons at 1, 2, 3, 4, 5, 6, 7, 8, 9, 10, 11, 12, 13, 14, 15, 16, 17, 18, 19, 20 or more codons in a single nucleic acid. In some instances, each variant long nucleic acid comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single long nucleic acid. In some instances, the variant nucleic acid population comprises variation for codons at 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more codons in a single nucleic acid. In some instances, the variant nucleic acid population comprises variation for codons in at least about 10, 20, 30, 40, 50, 60, 70, 80, 90, 100 or more codons in a single long nucleic acid.

### Highly Parallel Nucleic Acid Synthesis

Provided herein is a platform approach utilizing miniaturization, parallelization, and vertical integration of the end-to-end process from polynucleotide synthesis to gene assembly within nanowells on silicon to create a revolutionary synthesis platform. Devices described herein provide, with the same footprint as a 96-well plate, a silicon synthesis platform capable of increasing throughput by a factor of up to 1,000 or more compared to traditional synthesis methods, with production of up to approximately 1,000,000 or more polynucleotides, or 10,000 or more genes in a single highly-parallelized run.

With the advent of next-generation sequencing, high resolution genomic data has become an important factor for studies that delve into the biological roles of various genes in both normal biology and disease pathogenesis. At the core of this research is the central dogma of molecular biology and the concept of “residue-by-residue transfer of sequential information.” Genomic information encoded in the DNA is transcribed into a message that is then translated into the protein that is the active product within a given biological pathway.

Another exciting area of study is on the discovery, development and manufacturing of therapeutic molecules focused on a highly-specific cellular target. High diversity DNA sequence libraries are at the core of development pipelines for targeted therapeutics. Gene mutants are used to express proteins in a design, build, and test protein engineering cycle that ideally culminates in an optimized gene for high expression of a protein with high affinity for its therapeutic target. As an example, consider the binding pocket of a receptor. The ability to test all sequence permutations of all residues within the binding pocket simultaneously will allow for a thorough exploration, increasing chances of success. Saturation mutagenesis, in which a researcher attempts to generate all possible mutations at a specific site within the receptor, represents one approach to this development challenge. Though costly and time- and labor-intensive, it enables each variant to be introduced into each position. In contrast, combinatorial mutagenesis, where a few selected positions or short stretch of DNA may be modified extensively, generates an incomplete repertoire of variants with biased representation.

To accelerate the drug development pipeline, a library with the desired variants available at the intended frequency in the right position available for testing—in other words, a precision library—enables reduced costs as well as turnaround time for screening. Provided herein are methods for synthesizing nucleic acid synthetic variant libraries which provide for precise introduction of each intended variant at the desired frequency. To the end user, this translates to the ability to not only thoroughly sample sequence space but also be able to query these hypotheses in an efficient manner, reducing cost and screening time. Genome-wide editing can elucidate important pathways, libraries where each variant and sequence permutation can be tested for optimal functionality, and thousands of genes can be used to reconstruct entire pathways and genomes to re-engineer biological systems for drug discovery.

In a first example, a drug itself can be optimized using methods described herein. For example, to improve a specified function of an antibody, a variant polynucleotide library encoding for a portion of the antibody is designed and synthesized. A variant nucleic acid library for the antibody can then be generated by processes described herein (e.g., PCR mutagenesis followed by insertion into a vector). The antibody is then expressed in a production cell line and screened for enhanced activity. Example screens include examining modulation in binding affinity to an antigen, stability, or effector function (e.g., ADCC, complement, or apoptosis). Exemplary regions to optimize the antibody include, without limitation, the Fc region, Fab region, variable region of the Fab region, constant region of the Fab region, variable domain of the heavy chain or light chain (V_{H} or V_{L}), and specific complementarity-determining regions (CDRs) of V_{H} or V_{L}.

Nucleic acid libraries synthesized by methods described herein may be expressed in various cells associated with a disease state. Cells associated with a disease state include cell lines, tissue samples, primary cells from a subject, cultured cells expanded from a subject, or cells in a model system. Exemplary model systems include, without limitation, plant and animal models of a disease state.

To identify a variant molecule associated with prevention, reduction or treatment of a disease state, a variant nucleic acid library described herein is expressed in a cell associated with a disease state, or one in which a cell a disease state can be induced. In some instances, an agent is used to induce a disease state in cells. Exemplary tools for disease state induction include, without limitation, a Cre/Lox recombination system, LPS inflammation induction, and streptozotocin to induce hypoglycemia. The cells associated with a disease state may be cells from a model system or cultured cells, as well as cells from a subject having a particular disease condition. Exemplary disease conditions include a bacterial, fungal, viral, autoimmune, or proliferative disorder (e.g., cancer). In some instances, the variant nucleic acid library is expressed in the model system, cell line, or primary cells derived from a subject, and screened for changes in at least one cellular activity. Exemplary cellular activities include, without limitation, proliferation, cycle progression, cell death, adhesion, migration, reproduction, cell signaling, energy production, oxygen utilization, metabolic activity, and aging, response to free radical damage, or any combination thereof.

### Substrates

Devices used as a surface for polynucleotide synthesis may be in the form of substrates which include, without limitation, homogenous array surfaces, patterned array surfaces, channels, beads, gels, and the like. Provided herein are substrates comprising a plurality of clusters, wherein each cluster comprises a plurality of loci that support the attachment and synthesis of polynucleotides. In some instances, substrates comprise a homogenous array surface. For example, the homogenous array surface is a homogenous plate. The term “locus” as used herein refers to a discrete region on a structure which provides support for polynucleotides encoding for a single predetermined sequence to extend from the surface. In some instances, a locus is on a two-dimensional surface, e.g., a substantially planar surface. In some instances, a locus is on a three-dimensional surface, e.g., a well, microwell, channel, or post. In some instances, a surface of a locus comprises a material that is actively functionalized to attach to at least one nucleotide for polynucleotide synthesis, or preferably, a population of identical nucleotides for synthesis of a population of polynucleotides. In some instances, polynucleotide refers to a population of polynucleotides encoding for the same nucleic acid sequence. In some cases, a surface of a substrate is inclusive of one or a plurality of surfaces of a substrate. The average error rates for polynucleotides synthesized within a library described here using the systems and methods provided are often less than 1 in 1000, less than about 1 in 2000, less than about 1 in 3000 or less often without error correction.

Provided herein are surfaces that support the parallel synthesis of a plurality of polynucleotides having different predetermined sequences at addressable locations on a common support. In some instances, a substrate provides support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more non-identical polynucleotides. In some cases, the surfaces provide support for the synthesis of more than 50, 100, 200, 400, 600, 800, 1000, 1200, 1400, 1600, 1800, 2,000; 5,000; 10,000; 20,000; 50,000; 100,000; 200,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; 10,000,000 or more polynucleotides encoding for distinct sequences. In some instances, at least a portion of the polynucleotides have an identical sequence or are configured to be synthesized with an identical sequence. In some instances, the substrate provides a surface environment for the growth of polynucleotides having at least 80, 90, 100, 120, 150, 175, 200, 225, 250, 275, 300, 325, 350, 375, 400, 425, 450, 475, 500 bases or more.

Provided herein are methods for polynucleotide synthesis on distinct loci of a substrate, wherein each locus supports the synthesis of a population of polynucleotides. In some cases, each locus supports the synthesis of a population of polynucleotides having a different sequence than a population of polynucleotides grown on another locus. In some instances, each polynucleotide sequence is synthesized with 1, 2, 3, 4, 5, 6, 7, 8, 9 or more redundancy across different loci within the same cluster of loci on a surface for polynucleotide synthesis. In some instances, the loci of a substrate are located within a plurality of clusters. In some instances, a substrate comprises at least 10, 500, 1000, 2000, 3000, 4000, 5000, 6000, 7000, 8000, 9000, 10000, 11000, 12000, 13000, 14000, 15000, 20000, 30000, 40000, 50000 or more clusters. In some instances, a substrate comprises more than 2,000; 5,000; 10,000; 100,000; 200,000; 300,000; 400,000; 500,000; 600,000; 700,000; 800,000; 900,000; 1,000,000; 1,100,000; 1,200,000; 1,300,000; 1,400,000; 1,500,000; 1,600,000; 1,700,000; 1,800,000; 1,900,000; 2,000,000; 300,000; 400,000; 500,000; 600,000; 700,000; 800,000; 900,000; 1,000,000; 1,200,000; 1,400,000; 1,600,000; 1,800,000; 2,000,000; 2,500,000; 3,000,000; 3,500,000; 4,000,000; 4,500,000; 5,000,000; or 10,000,000 or more distinct loci. In some instances, a substrate comprises about 10,000 distinct loci. The amount of loci within a single cluster is varied in different instances. In some cases, each cluster includes 1, 2, 3, 4, 5, 6, 7, 8, 9, 10, 20, 30, 40, 50, 60, 70, 80, 90, 100, 120, 130, 150, 200, 300, 400, 500 or more loci. In some instances, each cluster includes about 50-500 loci. In some instances, each cluster includes about 100-200 loci. In some instances, each cluster includes about 100-150 loci. In some instances, each cluster includes about 109, 121, 130 or 137 loci. In some instances, each cluster includes about 19, 20, 61, 64 or more loci. Alternatively or in combination, polynucleotide synthesis occurs on a homogenous array surface.

In some instances, the number of distinct polynucleotides synthesized on a substrate is dependent on the number of distinct loci available in the substrate. In some instances, the density of loci within a cluster or surface of a substrate is at least or about 1, 10, 25, 50, 65, 75, 100, 130, 150, 175, 200, 300, 400, 500, 1,000 or more loci per mm². In some cases, a substrate comprises 10-500, 25-400, 50-500, 100-500, 150-500, 10-250, 50-250, 10-200, or 50-200 mm². In some instances, the distance between the centers of two adjacent loci within a cluster or surface is from about 10-500, from about 10-200, or from about 10-100 um. In some instances, the distance between two centers of adjacent loci is greater than about 10, 20, 30, 40, 50, 60, 70, 80, 90 or 100 μm. In some instances, the distance between the centers of two adjacent loci is less than about 200, 150, 100, 80, 70, 60, 50, 40, 30, 20 or 10 μm. In some instances, each locus has a width of about 0.5, 1, 2, 3, 4, 5, 6, 7, 8, 9, 10, 20, 30, 40, 50, 60, 70, 80, 90 or 100 μm. In some cases, each locus has a width of about 0.5-100, 0.5-50, 10-75, or 0.5-50 um.

In some instances, the density of clusters within a substrate is at least or about 1 cluster per 100 mm², 1 cluster per 10 mm², 1 cluster per 5 mm², 1 cluster per 4 mm², 1 cluster per 3 mm², 1 cluster per 2 mm², 1 cluster per 1 mm², 2 clusters per 1 mm², 3 clusters per 1 mm², 4 clusters per 1 mm², 5 clusters per 1 mm², 10 clusters per 1 mm², 50 clusters per 1 mm² or more. In some instances, a substrate comprises from about 1 cluster per 10 mm² to about 10 clusters per 1 mm². In some instances, the distance between the centers of two adjacent clusters is at least or about 50, 100, 200, 500, 1000, 2000, or 5000 μm. In some cases, the distance between the centers of two adjacent clusters is between about 50-100, 50-200, 50-300, 50-500, and 100-2000 um. In some cases, the distance between the centers of two adjacent clusters is between about 0.05-50, 0.05-10, 0.05-5, 0.05-4, 0.05-3, 0.05-2, 0.1-10, 0.2-10, 0.3-10, 0.4-10, 0.5-10, 0.5-5, or 0.5-2 mm. In some cases, each cluster has a cross section of about 0.5 to about 2, about 0.5 to about 1, or about 1 to about 2 mm. In some cases, each cluster has a cross section of about 0.5, 0.6, 0.7, 0.8, 0.9, 1, 1.1, 1.2, 1.3, 1.4, 1.5, 1.6, 1.7, 1.8, 1.9 or 2 mm. In some cases, each cluster has an interior cross section of about 0.5, 0.6, 0.7, 0.8, 0.9, 1, 1.1, 1.15, 1.2, 1.3, 1.4, 1.5, 1.6, 1.7, 1.8, 1.9 or 2 mm.

In some instances, a substrate is about the size of a standard 96 well plate, for example between about 100 and about 200 mm by between about 50 and about 150 mm. In some instances, a substrate has a diameter less than or equal to about 1000, 500, 450, 400, 300, 250, 200, 150, 100 or 50 mm. In some instances, the diameter of a substrate is between about 25-1000, 25-800, 25-600, 25-500, 25-400, 25-300, or 25-200 mm. In some instances, a substrate has a planar surface area of at least about 100; 200; 500; 1,000; 2,000; 5,000; 10,000; 12,000; 15,000; 20,000; 30,000; 40,000; 50,000 mm² or more. In some instances, the thickness of a substrate is between about 50-2000, 50-1000, 100-1000, 200-1000, or 250-1000 mm.

### Surface Materials

Substrates, devices, and reactors provided herein are fabricated from any variety of materials suitable for the methods, compositions, and systems described herein. In certain instances, substrate materials are fabricated to exhibit a low level of nucleotide binding. In some instances, substrate materials are modified to generate distinct surfaces that exhibit a high level of nucleotide binding. In some instances, substrate materials are transparent to visible and/or UV light. In some instances, substrate materials are sufficiently conductive, e.g., are able to form uniform electric fields across all or a portion of a substrate. In some instances, conductive materials are connected to an electric ground. In some instances, the substrate is heat conductive or insulated. In some instances, the materials are chemical resistant and heat resistant to support chemical or biochemical reactions, for example polynucleotide synthesis reaction processes. In some instances, a substrate comprises flexible materials. For flexible materials, materials can include, without limitation: nylon, both modified and unmodified, nitrocellulose, polypropylene, and the like. In some instances, a substrate comprises rigid materials. For rigid materials, materials can include, without limitation: glass; fuse silica; silicon, plastics (for example polytetraflouroethylene, polypropylene, polystyrene, polycarbonate, and blends thereof, and the like); and metals (for example, gold, platinum, and the like). The substrate, solid support or reactors can be fabricated from a material selected from the group consisting of silicon, polystyrene, agarose, dextran, cellulosic polymers, polyacrylamides, polydimethylsiloxane (PDMS), and glass. The substrates/solid supports or the microstructures/reactors therein may be manufactured with a combination of materials listed herein or any other suitable material known in the art.

### Surface Architecture

Provided herein are substrates for the methods, compositions, and systems described herein, wherein the substrates have a surface architecture suitable for the methods, compositions, and systems described herein. In some instances, a substrate comprises raised and/or lowered features. One benefit of having such features is an increase in surface area to support polynucleotide synthesis. In some instances, a substrate having raised and/or lowered features is referred to as a three-dimensional substrate. In some cases, a three-dimensional substrate comprises one or more channels. In some cases, one or more loci comprise a channel. In some cases, the channels are accessible to reagent deposition via a deposition device such as a material deposition device. In some cases, reagents and/or fluids collect in a larger well in fluid communication one or more channels. For example, a substrate comprises a plurality of channels corresponding to a plurality of loci with a cluster, and the plurality of channels are in fluid communication with one well of the cluster. In some methods, a library of polynucleotides is synthesized in a plurality of loci of a cluster.

Provided herein are substrates for the methods, compositions, and systems described herein, wherein the substrates are configured for polynucleotide synthesis. In some instances, the structure is configured to allow for controlled flow and mass transfer paths for polynucleotide synthesis on a surface. In some instances, the configuration of a substrate allows for the controlled and even distribution of mass transfer paths, chemical exposure times, and/or wash efficacy during polynucleotide synthesis. In some instances, the configuration of a substrate allows for increased sweep efficiency, for example by providing sufficient volume for a growing polynucleotide such that the excluded volume by the growing polynucleotide does not take up more than 50, 45, 40, 35, 30, 25, 20, 15, 14, 13, 12, 11, 10, 9, 8, 7, 6, 5, 4, 3, 2, 1%, or less of the initially available volume that is available or suitable for growing the polynucleotide. In some instances, a three-dimensional structure allows for managed flow of fluid to allow for the rapid exchange of chemical exposure.

Provided herein are substrates for the methods, compositions, and systems described herein, wherein the substrates comprise structures suitable for the methods, compositions, and systems described herein. In some instances, segregation is achieved by physical structure. In some instances, segregation is achieved by differential functionalization of the surface generating active and passive regions for polynucleotide synthesis. In some instances, differential functionalization is achieved by alternating the hydrophobicity across the substrate surface, thereby creating water contact angle effects that cause beading or wetting of the deposited reagents. Employing larger structures can decrease splashing and cross-contamination of distinct polynucleotide synthesis locations with reagents of the neighboring spots. In some cases, a device, such as a material deposition device, is used to deposit reagents to distinct polynucleotide synthesis locations. Substrates having three-dimensional features are configured in a manner that allows for the synthesis of a large number of polynucleotides (e.g., more than about 10,000) with a low error rate (e.g., less than about 1:500, 1:1000, 1:1500, 1:2,000, 1:3,000, 1:5,000, or 1:10,000). In some cases, a substrate comprises features with a density of about or greater than about 1, 5, 10, 20, 30, 40, 50, 60, 70, 80, 100, 110, 120, 130, 140, 150, 160, 170, 180, 190, 200, 300, 400 or 500 features per mm².

A well of a substrate may have the same or different width, height, and/or volume as another well of the substrate. A channel of a substrate may have the same or different width, height, and/or volume as another channel of the substrate. In some instances, the diameter of a cluster or the diameter of a well comprising a cluster, or both, is between about 0.05-50, 0.05-10, 0.05-5, 0.05-4, 0.05-3, 0.05-2, 0.05-1, 0.05-0.5, 0.05-0.1, 0.1-10, 0.2-10, 0.3-10, 0.4-10, 0.5-10, 0.5-5, or 0.5-2 mm. In some instances, the diameter of a cluster or well or both is less than or about 5, 4, 3, 2, 1, 0.5, 0.1, 0.09, 0.08, 0.07, 0.06, or 0.05 mm. In some instances, the diameter of a cluster or well or both is between about 1.0 and 1.3 mm. In some instances, the diameter of a cluster or well, or both is about 1.150 mm. In some instances, the diameter of a cluster or well, or both is about 0.08 mm. The diameter of a cluster refers to clusters within a two-dimensional or three-dimensional substrate.

In some instances, the height of a well is from about 20-1000, 50-1000, 100-1000, 200-1000, 300-1000, 400-1000, or 500-1000 um. In some cases, the height of a well is less than about 1000, 900, 800, 700, or 600 um.

In some instances, a substrate comprises a plurality of channels corresponding to a plurality of loci within a cluster, wherein the height or depth of a channel is 5-500, 5-400, 5-300, 5-200, 5-100, 5-50, or 10-50 um. In some cases, the height of a channel is less than 100, 80, 60, 40, or 20 um.

In some instances, the diameter of a channel, locus (e.g., in a substantially planar substrate) or both channel and locus (e.g., in a three-dimensional substrate wherein a locus corresponds to a channel) is from about 1-1000, 1-500, 1-200, 1-100, 5-100, or 10-100 um, for example, to about 90, 80, 70, 60, 50, 40, 30, 20 or 10 μm. In some instances, the diameter of a channel, locus, or both channel and locus is less than about 100, 90, 80, 70, 60, 50, 40, 30, 20 or 10 μm. In some instances, the distance between the center of two adjacent channels, loci, or channels and loci is from about 1-500, 1-200, 1-100, 5-200, 5-100, 5-50, or 5-30, for example, to about 20 um.

### Surface Modifications

Provided herein are methods for polynucleotide synthesis on a surface, wherein the surface comprises various surface modifications. In some instances, the surface modifications are employed for the chemical and/or physical alteration of a surface by an additive or subtractive process to change one or more chemical and/or physical properties of a substrate surface or a selected site or region of a substrate surface. For example, surface modifications include, without limitation, (1) changing the wetting properties of a surface, (2) functionalizing a surface, i.e., providing, modifying or substituting surface functional groups, (3) defunctionalizing a surface, i.e., removing surface functional groups, (4) otherwise altering the chemical composition of a surface, e.g., through etching, (5) increasing or decreasing surface roughness, (6) providing a coating on a surface, e.g., a coating that exhibits wetting properties that are different from the wetting properties of the surface, and/or (7) depositing particulates on a surface.

In some cases, the addition of a chemical layer on top of a surface (referred to as adhesion promoter) facilitates structured patterning of loci on a surface of a substrate. Exemplary surfaces for application of adhesion promotion include, without limitation, glass, silicon, silicon dioxide and silicon nitride. In some cases, the adhesion promoter is a chemical with a high surface energy. In some instances, a second chemical layer is deposited on a surface of a substrate. In some cases, the second chemical layer has a low surface energy. In some cases, surface energy of a chemical layer coated on a surface supports localization of droplets on the surface. Depending on the patterning arrangement selected, the proximity of loci and/or area of fluid contact at the loci are alterable.

In some instances, a substrate surface, or resolved loci, onto which nucleic acids or other moieties are deposited, e.g., for polynucleotide synthesis, are smooth or substantially planar (e.g., two-dimensional) or have irregularities, such as raised or lowered features (e.g., three-dimensional features). In some instances, a substrate surface is modified with one or more different layers of compounds. Such modification layers of interest include, without limitation, inorganic and organic layers such as metals, metal oxides, polymers, small organic molecules, and the like.

In some instances, resolved loci of a substrate are functionalized with one or more moieties that increase and/or decrease surface energy. In some cases, a moiety is chemically inert. In some cases, a moiety is configured to support a desired chemical reaction, for example, one or more processes in a polynucleotide synthesis reaction. The surface energy, or hydrophobicity, of a surface is a factor for determining the affinity of a nucleotide to attach onto the surface. In some instances, a method for substrate functionalization comprises: (a) providing a substrate having a surface that comprises silicon dioxide; and (b) silanizing the surface using a suitable silanizing agent described herein or otherwise known in the art, for example, an organofunctional alkoxysilane molecule. Methods and functionalizing agents are described in U.S. Pat. No. 5,474,796, which is herein incorporated by reference in its entirety.

In some instances, a substrate surface is functionalized by contact with a derivatizing composition that contains a mixture of silanes, under reaction conditions effective to couple the silanes to the substrate surface, typically via reactive hydrophilic moieties present on the substrate surface. Silanization generally covers a surface through self-assembly with organofunctional alkoxysilane molecules. A variety of siloxane functionalizing reagents can further be used as currently known in the art, e.g., for lowering or increasing surface energy. The organofunctional alkoxysilanes are classified according to their organic functions.

### Polynucleotide Synthesis

Methods of the current disclosure for polynucleotide synthesis may include processes involving phosphoramidite chemistry. In some instances, polynucleotide synthesis comprises coupling a base with phosphoramidite. Polynucleotide synthesis may comprise coupling a base by deposition of phosphoramidite under coupling conditions, wherein the same base is optionally deposited with phosphoramidite more than once, i.e., double coupling. Polynucleotide synthesis may comprise capping of unreacted sites. In some instances, capping is optional. Polynucleotide synthesis may also comprise oxidation or an oxidation step or oxidation steps. Polynucleotide synthesis may comprise deblocking, detritylation, and sulfurization. In some instances, polynucleotide synthesis comprises either oxidation or sulfurization. In some instances, between one or each step during a polynucleotide synthesis reaction, the device is washed, for example, using tetrazole or acetonitrile. Time frames for any one step in a phosphoramidite synthesis method may be less than about 2 min, 1 min, 50 sec, 40 sec, 30 sec, 20 sec and 10 sec.

Polynucleotide synthesis using a phosphoramidite method may comprise a subsequent addition of a phosphoramidite building block (e.g., nucleoside phosphoramidite) to a growing polynucleotide chain for the formation of a phosphite triester linkage. Phosphoramidite polynucleotide synthesis proceeds in the 3′ to 5′ direction. Phosphoramidite polynucleotide synthesis allows for the controlled addition of one nucleotide to a growing nucleic acid chain per synthesis cycle. In some instances, each synthesis cycle comprises a coupling step. Phosphoramidite coupling involves the formation of a phosphite triester linkage between an activated nucleoside phosphoramidite and a nucleoside bound to the substrate, for example, via a linker. In some instances, the nucleoside phosphoramidite is provided to the device activated. In some instances, the nucleoside phosphoramidite is provided to the device with an activator. In some instances, nucleoside phosphoramidites are provided to the device in a 1.5, 2, 3, 4, 5, 6, 7, 8, 9, 10, 11, 12, 13, 14, 15, 16, 17, 18, 19, 20, 25, 30, 35, 40, 50, 60, 70, 80, 90, 100-fold excess or more over the substrate-bound nucleosides. In some instances, the addition of nucleoside phosphoramidite is performed in an anhydrous environment, for example, in anhydrous acetonitrile. Following addition of a nucleoside phosphoramidite, the device is optionally washed. In some instances, the coupling step is repeated one or more additional times, optionally with a wash step between nucleoside phosphoramidite additions to the substrate. In some instances, a polynucleotide synthesis method used herein comprises 1, 2, 3 or more sequential coupling steps. Prior to coupling, in many cases, the nucleoside bound to the device is de-protected by removal of a protecting group, where the protecting group functions to prevent polymerization. A common protecting group is 4,4′-dimethoxytrityl (DMT).

Following coupling, phosphoramidite polynucleotide synthesis methods optionally comprise a capping step. In a capping step, the growing polynucleotide is treated with a capping agent. A capping step is useful to block unreacted substrate-bound 5′-OH groups after coupling from further chain elongation, preventing the formation of polynucleotides with internal base deletions. Further, phosphoramidites activated with 1H-tetrazole may react, to a small extent, with the O6 position of guanosine. Without being bound by theory, upon oxidation with I₂/water, this side product, possibly via O6-N7 migration, may undergo depurination. The apurinic sites may end up being cleaved in the course of the final deprotection of the polynucleotide thus reducing the yield of the full-length product. The O6 modifications may be removed by treatment with the capping reagent prior to oxidation with I₂/water. In some instances, inclusion of a capping step during polynucleotide synthesis decreases the error rate as compared to synthesis without capping. As an example, the capping step comprises treating the substrate-bound polynucleotide with a mixture of acetic anhydride and 1-methylimidazole. Following a capping step, the device is optionally washed.

In some instances, following addition of a nucleoside phosphoramidite, and optionally after capping and one or more wash steps, the device bound growing nucleic acid is oxidized. The oxidation step comprises a phosphite triester which is oxidized into a tetracoordinated phosphate triester, a protected precursor of the naturally occurring phosphate diester internucleoside linkage. In some instances, oxidation of the growing polynucleotide is achieved by treatment with iodine and water, optionally in the presence of a weak base (e.g., pyridine, lutidine, collidine). Oxidation may be carried out under anhydrous conditions using, e.g. tert-Butyl hydroperoxide or (1S)(+)-(10-camphorsulfonyl)-oxaziridine (CSO). In some methods, a capping step is performed following oxidation. A second capping step allows for device drying, as residual water from oxidation that may persist can inhibit subsequent coupling. Following oxidation, the device and growing polynucleotide are optionally washed. In some instances, the step of oxidation is substituted with a sulfurization step to obtain polynucleotide phosphorothioates, wherein any capping steps can be performed after the sulfurization. Many reagents are capable of the efficient sulfur transfer, including but not limited to 3-(Dimethylaminomethylidene)amino)-3H-1,2,4-dithiazole-3-thione, DDTT, 3H-1,2-benzodithiol-3-one 1,1-dioxide, also known as Beaucage reagent, and N,N,N′N′-Tetraethylthiuram disulfide (TETD).

In order for a subsequent cycle of nucleoside incorporation to occur through coupling, the protected 5′ end of the device bound growing polynucleotide is removed so that the primary hydroxyl group is reactive with a next nucleoside phosphoramidite. In some instances, the protecting group is DMT and deblocking occurs with trichloroacetic acid in dichloromethane. Conducting detritylation for an extended time or with stronger than recommended solutions of acids may lead to increased depurination of solid support-bound polynucleotide and thus reduces the yield of the desired full-length product. Methods and compositions of the disclosure described herein provide for controlled deblocking conditions limiting undesired depurination reactions. In some instances, the device bound polynucleotide is washed after deblocking. In some instances, efficient washing after deblocking contributes to synthesized polynucleotides having a low error rate.

Methods for the synthesis of polynucleotides typically involve an iterating sequence of the following steps: application of a protected monomer to an actively functionalized surface (e.g., locus) to link with either the activated surface, a linker or with a previously deprotected monomer; deprotection of the applied monomer so that it is reactive with a subsequently applied protected monomer; and application of another protected monomer for linking. One or more intermediate steps include oxidation or sulfurization. In some instances, one or more wash steps precede or follow one or all of the steps.

Methods for phosphoramidite-based polynucleotide synthesis comprise a series of chemical steps. In some instances, one or more steps of a synthesis method involve reagent cycling, where one or more steps of the method comprise application to the device of a reagent useful for the step. For example, reagents are cycled by a series of liquid deposition and vacuum drying steps. For substrates comprising three-dimensional features such as wells, microwells, channels and the like, reagents are optionally passed through one or more regions of the device via the wells and/or channels.

Methods and systems described herein relate to polynucleotide synthesis devices for the synthesis of polynucleotides. The synthesis may be in parallel. For example, at least or about at least 2, 3, 4, 5, 6, 7, 8, 9, 10, 11, 12, 13, 14, 15, 16, 17, 18, 19, 20, 21, 22, 23, 24, 25, 30, 35, 40, 45, 50, 100, 150, 200, 250, 300, 350, 400, 450, 500, 550, 600, 650, 700, 750, 800, 850, 900, 1000, 10000, 50000, 75000, 100000 or more polynucleotides can be synthesized in parallel. The total number polynucleotides that may be synthesized in parallel may be from 2-100000, 3-50000, 4-10000, 5-1000, 6-900, 7-850, 8-800, 9-750, 10-700, 11-650, 12-600, 13-550, 14-500, 15-450, 16-400, 17-350, 18-300, 19-250, 20-200, 21-150,22-100, 23-50, 24-45, 25-40, 30-35. Those of skill in the art appreciate that the total number of polynucleotides synthesized in parallel may fall within any range bound by any of these values, for example 25-100. The total number of polynucleotides synthesized in parallel may fall within any range defined by any of the values serving as endpoints of the range. Total molar mass of polynucleotides synthesized within the device or the molar mass of each of the polynucleotides may be at least or at least about 10, 20, 30, 40, 50, 100, 250, 500, 750, 1000, 2000, 3000, 4000, 5000, 6000, 7000, 8000, 9000, 10000, 25000, 50000, 75000, 100000 picomoles, or more. The length of each of the polynucleotides or average length of the polynucleotides within the device may be at least or about at least 10, 15, 20, 25, 30, 35, 40, 45, 50, 100, 150, 200, 300, 400, 500 nucleotides, or more. The length of each of the polynucleotides or average length of the polynucleotides within the device may be at most or about at most 500, 400, 300, 200, 150, 100, 50, 45, 35, 30, 25, 20, 19, 18, 17, 16, 15, 14, 13, 12, 11, 10 nucleotides, or less. The length of each of the polynucleotides or average length of the polynucleotides within the device may fall from 10-500, 9-400, 11-300, 12-200, 13-150, 14-100, 15-50, 16-45, 17-40, 18-35, 19-25. Those of skill in the art appreciate that the length of each of the polynucleotides or average length of the polynucleotides within the device may fall within any range bound by any of these values, for example 100-300. The length of each of the polynucleotides or average length of the polynucleotides within the device may fall within any range defined by any of the values serving as endpoints of the range.

Methods for polynucleotide synthesis on a surface provided herein allow for synthesis at a fast rate. As an example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 70, 80, 90, 100, 125, 150, 175, 200 nucleotides per hour, or more are synthesized. Nucleotides include adenine, guanine, thymine, cytosine, uridine building blocks, or analogs/modified versions thereof. In some instances, libraries of polynucleotides are synthesized in parallel on substrate. For example, a device comprising about or at least about 100; 1,000; 10,000; 30,000; 75,000; 100,000; 1,000,000; 2,000,000; 3,000,000; 4,000,000; or 5,000,000 resolved loci is able to support the synthesis of at least the same number of distinct polynucleotides, wherein polynucleotide encoding a distinct sequence is synthesized on a resolved locus. In some instances, a library of polynucleotides is synthesized on a device with low error rates described herein in less than about three months, two months, one month, three weeks, 15, 14, 13, 12, 11, 10, 9, 8, 7, 6, 5, 4, 3, 2 days, 24 hours, or less. In some instances, larger nucleic acids assembled from a polynucleotide library synthesized with low error rate using the substrates and methods described herein are prepared in less than about three months, two months, one month, three weeks, 15, 14, 13, 12, 11, 10, 9, 8, 7, 6, 5, 4, 3, 2 days, 24 hours, or less.

In some instances, methods described herein provide for generation of a library of nucleic acids comprising variant nucleic acids differing at a plurality of codon sites. In some instances, a nucleic acid may have 1 site, 2 sites, 3 sites, 4 sites, 5 sites, 6 sites, 7 sites, 8 sites, 9 sites, 10 sites, 11 sites, 12 sites, 13 sites, 14 sites, 15 sites, 16 sites, 17 sites 18 sites, 19 sites, 20 sites, 30 sites, 40 sites, 50 sites, or more of variant codon sites.

In some instances, the one or more sites of variant codon sites may be adjacent. In some instances, the one or more sites of variant codon sites may not be adjacent but are separated by 1, 2, 3, 4, 5, 6, 7, 8, 9, 10, or more codons.

In some instances, a nucleic acid may comprise multiple sites of variant codon sites, wherein all the variant codon sites are adjacent to one another, forming a stretch of variant codon sites. In some instances, a nucleic acid may comprise multiple sites of variant codon sites, wherein none the variant codon sites are adjacent to one another. In some instances, a nucleic acid may comprise multiple sites of variant codon sites, wherein some the variant codon sites are adjacent to one another, forming a stretch of variant codon sites, and some of the variant codon sites are not adjacent to one another.

An exemplary process workflow for synthesis of nucleic acids (e.g., genes) from shorter nucleic acids may used for synthesis of polynucleotides described herein. The workflow is divided generally into phases: (1) de novo synthesis of a single stranded nucleic acid library, (2) joining nucleic acids to form larger fragments, (3) error correction, (4) quality control, and (5) shipment. Prior to de novo synthesis, an intended nucleic acid sequence or group of nucleic acid sequences is preselected. For example, a group of genes is preselected for generation.

Once large nucleic acids for generation are selected, a predetermined library of nucleic acids is designed for de novo synthesis. Various suitable methods are known for generating high density polynucleotide arrays. In the workflow example, a device surface layer is provided. In the example, chemistry of the surface is altered in order to improve the polynucleotide synthesis process. Areas of low surface energy are generated to repel liquid while areas of high surface energy are generated to attract liquids. The surface itself may be in the form of a planar surface or contain variations in shape, such as protrusions or microwells which increase surface area. In the workflow example, high surface energy molecules selected serve a dual function of supporting DNA chemistry, as disclosed in International Patent Application Publication WO/2015/021080, which is herein incorporated by reference in its entirety.

In situ preparation of polynucleotide arrays is generated on a solid support and utilizes single nucleotide extension process to extend multiple oligomers in parallel. A deposition device, such as a material deposition device, is designed to release reagents in a step-wise fashion such that multiple polynucleotides extend, in parallel, one residue at a time to generate oligomers with a predetermined nucleic acid sequence. In some instances, polynucleotides are cleaved from the surface at this stage. Cleavage includes gas cleavage, e.g., with ammonia or methylamine.

The generated polynucleotide libraries are placed in a reaction chamber. In this exemplary workflow, the reaction chamber (also referred to as “nanoreactor”) is a silicon coated well, containing PCR reagents and lowered onto the polynucleotide library. Prior to or after the sealing of the polynucleotides, a reagent is added to release the polynucleotides from the substrate. In the exemplary workflow, the polynucleotides are released subsequent to sealing of the nanoreactor. Once released, fragments of single stranded polynucleotides hybridize in order to span an entire long-range sequence of DNA. Partial hybridization is possible because each synthesized polynucleotide is designed to have a small portion overlapping with at least one other polynucleotide in the pool.

After hybridization, a PCA reaction is commenced. During the polymerase cycles, the polynucleotides anneal to complementary fragments and gaps are filled in by a polymerase. Each cycle increases the length of various fragments randomly depending on which polynucleotides find each other. Complementarity amongst the fragments allows for formation of a complete large span of double stranded DNA.

After PCA is complete, the nanoreactor is separated from the device and positioned for interaction with a device having primers for PCR. After sealing, the nanoreactor is subject to PCR and the larger nucleic acids are amplified. After PCR, the nanochamber is opened, error correction reagents are added, the chamber is sealed and an error correction reaction occurs to remove mismatched base pairs and/or strands with poor complementarity from the double stranded PCR amplification products. The nanoreactor is opened and separated. Error corrected product is next subject to additional processing steps, such as PCR and molecular bar coding, and then packaged for shipment.

In some instances, quality control measures are taken. After error correction, quality control steps include for example interaction with a wafer having sequencing primers for amplification of the error corrected product, sealing the wafer to a chamber containing error corrected amplification product, and performing an additional round of amplification. The nanoreactor is opened and the products are pooled and sequenced. After an acceptable quality control determination is made, the packaged product is approved for shipment.

In some instances, a nucleic acid generated by a workflow is subject to mutagenesis using overlapping primers disclosed herein. In some instances, a library of primers is generated by in situ preparation on a solid support and utilize single nucleotide extension process to extend multiple oligomers in parallel. A deposition device, such as a material deposition device, is designed to release reagents in a step wise fashion such that multiple polynucleotides extend, in parallel, one residue at a time to generate oligomers with a predetermined nucleic acid sequence.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1

Wnt signaling plays an important role in embryonic development and tumorigenesis. These biological effects are exerted by activation of the f-catenin/TCF transcription complex and consequent regulation of a set of downstream genes. DKK1 has been shown to be a potent inhibitor of Wnt signaling via competing Wnt binding to LDL Receptor-related Protein 5/6 (LRP5/6). DKK1 is tumorigenic in multiple cancer types and also immunosuppressive via MDSCs and NIK cells. Emerging evidence indicates that DKK1 has been involved in T cell differentiation and induction of cancer evasion of immune surveillance by accumulating MDSCs. Consequently, DKK1 has become a promising target for cancer immunotherapy, and the mechanisms of DKK1 affecting cancers and immune cells have received great attention.

Phage display libraries were created (Twist Bioscience) with diversity greater than 1×10¹⁰, and utilized machine learning model for optimal discovery (Table 4).

TABLE 4

| | | Refor- | | | | |
|---|---|---|---|---|---|---|
| Proj- | | matted, | TCF | Immune | Tumor | In |
| ect | Library | SPR | signaling | activation | killing | vivo |
| 1 | VHH | 113 | 5 | 4 | 4 | 1 |
| | hShuffle, | | | | | |
| | VHH | | | | | |
| | Hyperimmune | | | | | |
| 2 | DeepCDR, | 109 | 4 | 2 | 2 | 0 |
| | Natural | | | | | |
| | Antibody | | | | | |
| | Library | | | | | |
| 3 | Hyperimmune | 97 | 2 | 0 | 0 | 0 |
| | Fab | | | | | |
| 4 | Synthetic | 40 | 5 | 1 | 1 | 2 |
| | Library A | | | | | |
| 5 | Synthetic | 200 | 15 | 6 | 6 | 0 |
| | Library B | | | | | |
| 6 | VHH | 97 | 12 | 8 | 6 | 1 |
| | hShuffle/HI | 170 | 14 | 15 | 9 | 0 |
| | Library | | | | | |

Amino acid sequences of exemplary DKK1 binders are shown in Table 5.

TABLE 5

| Name | SEQ ID | Sequence |
|---|---|---|
| DKK1-133 | 1 | EVQLVESGGGLVQPGGSLRLSCAASGRTFTMGWFRQAPGKERELVAAINRSGK |
| (linker | | STYYADSVKGRFTISADNSKNTAYLQMNSLKPEDTAVYYCAAGNGGRHYGHSR |
| between VHH | | ARYDWGQGTLVTVSSGGGGSVECPPCPAPPVAGPSVFLFPPKPKDTLMISRTPEV |
| and Fc region | | TCVVVDVSHEDPEVQFNWYVDGVEVHNAKTKPREEQFNSTFRVVSVLTVVHQ |
| is underlined) | | DWLNGKEYKCKVSNKGLPAPIEKTISKTKGQPREPQVYTLPPSREEMTKNQVSL |
| | | TCLVKGFYPSDIAVEWESNGQPENNYKTTPPMLDSDGSFFLYSKLTVDKSRWQQ |
| | | GNVFSCSVMHEALHNHYTQKSLSLSPG |
| DKK1-133 | 20 | EVQLVESGGGLVQPGGSLRLSCAASGRTFTMGWFRQAPGKERELVAAINRSGK |
| variable | | STYYADSVKGRFTISADNSKNTAYLQMNSLKPEDTAVYYCAAGNGGRHYGHSR |
| heavy chain | | ARYDWGQGTLVTVSS |
| DKK1-134 | 2 | EVQLVESGGGLVQPGGSLRLSCAASGRGAMGWFRQAPGKEREFVAAINRSGKS |
| (linker | | TYYADSVKGRFTISADNSKNTAYLQMNSLKPEDTAVYYCAAGNGGRSYGHSRA |
| between VHH | | RYDWGQGTLVTVSSGGGGSVECPPCPAPPVAGPSVFLFPPKPKDTLMISRTPEVT |
| and Fc region | | CVVVDVSHEDPEVQFNWYVDGVEVHNAKTKPREEQFNSTFRVVSVLTVVHQD |
| is underlined) | | WLNGKEYKCKVSNKGLPAPIEKTISKTKGQPREPQVYTLPPSREEMTKNQVSLT |
| | | CLVKGFYPSDIAVEWESNGQPENNYKTTPPMLDSDGSFFLYSKLTVDKSRWQQ |
| | | GNVFSCSVMHEALHNHYTQKSLSLSPG |
| DKK1-134 | 21 | EVQLVESGGGLVQPGGSLRLSCAASGRGAMGWFRQAPGKEREFVAAINRSGKS |
| variable | | TYYADSVKGRFTISADNSKNTAYLQMNSLKPEDTAVYYCAAGNGGRSYGHSRA |
| heavy chain | | RYDWGQGTLVTVSS |
| DKK1-135 | 3 | EVQLVESGGGLVQPGGSLRLSCAASGRTFSNFAMGWFRQAPGKEREFVAVILRG |
| (linkers | | GSTNYADSVKGRFTISADNSKNTAYLQMNSLKPEDTAVYYCAAAPRPKRVSVS |
| between | | YFSTASNYDWGQGTLVTVSSGGGGSVECPPCPAPPVAGPSVFLFPPKPKDTLMIS |
| VHHs and Fc | | RTPEVTCVVVDVSHEDPEVQFNWYVDGVEVHNAKTKPREEQFNSTFRVVSVLT |
| region are | | VVHQDWLNGKEYKCKVSNKGLPAPIEKTISKTKGQPREPQVYTLPPSREEMTKN |
| underlined) | | QVSLTCLVKGFYPSDIAVEWESNGQPENNYKTTPPMLDSDGSFFLYSKLTVDKS |
| | | RWQQGNVFSCSVMHEALHNHYTQKSLSLSPGGGGGSGGGGSASEVQLVESGG |
| | | GLVQPGGSLRLSCAASGRIFGGYAMGWFRQAPGKEREFVAAISWSGASAIYADS |
| | | VKGRFTISADNSKNTAYLQMNSLKPEDTAVYYCAAGNGGSRYGHSRARYDWG |
| | | QGTLVTVSS |
| DKK1-136 | 4 | EVQLVESGGGLVQPGGSLRLSCAASGNIDRLYAMGWFRQAPGKEREFVAAISW |
| (linkers | | SVSSTYYADSVKGRFTISADNSKNTAYLQMNSLKPEDTAVYYCAAEGGYSGTY |
| between | | YYTGDFDWGQGTLVTVSSGGGGSVECPPCPAPPVAGPSVFLFPPKPKDTLMISRT |
| VHHs and Fc | | PEVTCVVVDVSHEDPEVQFNWYVDGVEVHNAKTKPREEQFNSTFRVVSVLTVV |
| region are | | HQDWLNGKEYKCKVSNKGLPAPIEKTISKTKGQPREPQVYTLPPSREEMTKNQV |
| underlined) | | SLTCLVKGFYPSDIAVEWESNGQPENNYKTTPPMLDSDGSFFLYSKLTVDKSRW |
| | | QQGNVFSCSVMHEALHNHYTQKSLSLSPGGGGGSGGGGSASEVQLVESGGGLV |
| | | QPGGSLRLSCAASGRIFGGYAMGWFRQAPGKEREFVAAISWSGASAIYADSVKG |
| | | RFTISADNSKNTAYLQMNSLKPEDTAVYYCAAGNGGSRYGHSRARYDWGQGT |
| | | LVTVSS |
| DKK1-135 | 22 | EVQLVESGGGLVQPGGSLRLSCAASGRTFSNFAMGWFRQAPGKEREFVAVILRG |
| variable | | GSTNYADSVKGRFTISADNSKNTAYLQMNSLKPEDTAVYYCAAAPRPKRVSVS |
| heavy chain 1 | | YFSTASNYDWGQGTLVTVSS |
| DKK1-136 | 23 | EVQLVESGGGLVQPGGSLRLSCAASGNIDRLYAMGWFRQAPGKEREFVAAISW |
| variable | | SVSSTYYADSVKGRFTISADNSKNTAYLQMNSLKPEDTAVYYCAAEGGYSGTY |
| heavy chain 1 | | YYTGDFDWGQGTLVTVSS |
| DKK1-135 | 24 | EVQLVESGGGLVQPGGSLRLSCAASGRIFGGYAMGWFRQAPGKEREFVAAISW |
| and | | SGASAIYADSVKGRFTISADNSKNTAYLQMNSLKPEDTAVYYCAAGNGGSRYG |
| DKK1-136 | | HSRARYDWGQGTLVTVSS |
| variable | | |
| heavy chain 2 | | |

CDR sequences of exemplary DKK1 binders are shown in Table 6.

TABLE 6
Variable Heavy Chain CDRs

| DKK1 | SEQ | CDR1 | SEQ ID | CDR2 | SEQ | CDR3 |
|---|---|---|---|---|---|---|
| Variant | ID NO | Sequence | NO | Sequence | ID NO | Sequence |
| DKK1-133 | 5 | GRTFT | 6 | INRSGKST | 7 | AAGNGGRHYGHSRARYD |
| DKK1-133 | | | 29 | NRSGKS | 30 | GNGGRHYGHSRARYD |
| alt. CDR2, | | | | | | |
| CDR3 | | | | | | |
| DKK1-134 | 8 | GRG | 9 | NRSGKS | 10 | GNGGRSYGHSRARYD |
| DKK1-135 | 11 | GRTFSNF | 12 | LRGGS | 13 | APRPKRVSVSYFSTA |
| VH1 | | | | | | SNYD |
| DKK1-135 | 14 | GRIFGGY | 15 | SWSGAS | 16 | GNGGSRYGHSRARYD |
| and | | | | | | |
| DKK1-136 | | | | | | |
| VH2 | | | | | | |
| DKK1-136 | 17 | GNIDRL Y | 18 | SWSVSS | 19 | EGGYSGTYYYTGDFD |
| VH1 | | | | | | |

Additional DKK1 binders were also evaluated (DKK1-1 to DKK1-17).

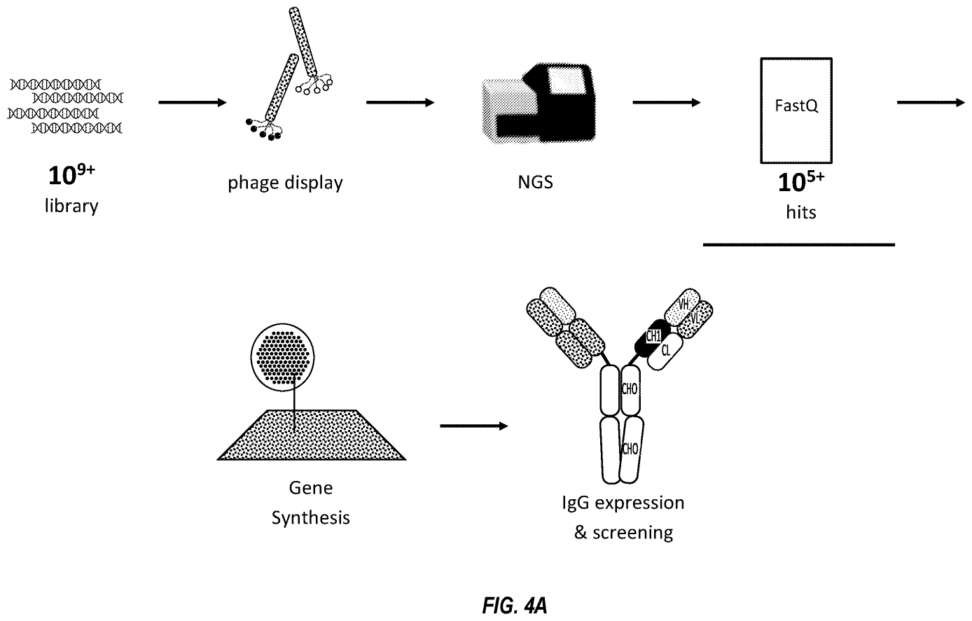

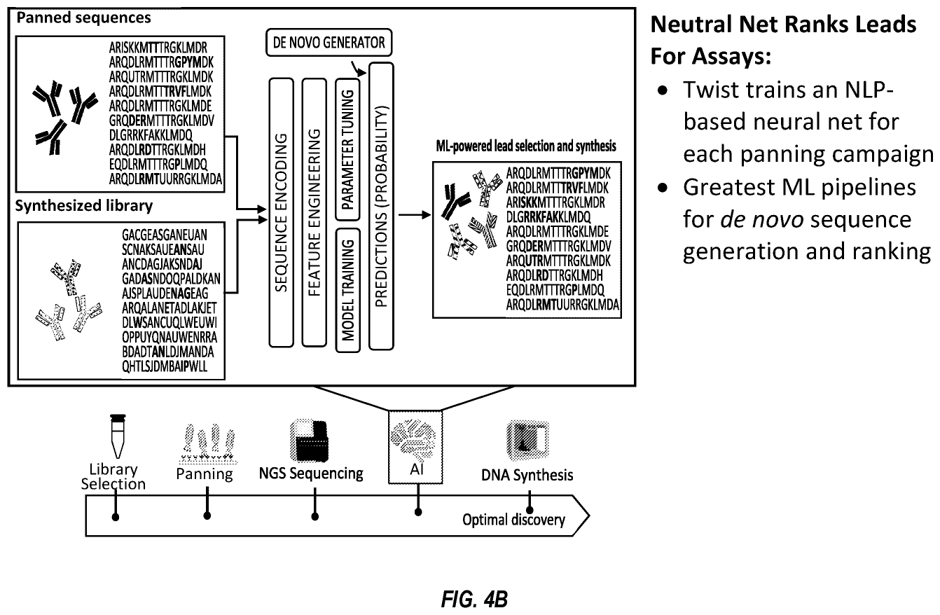

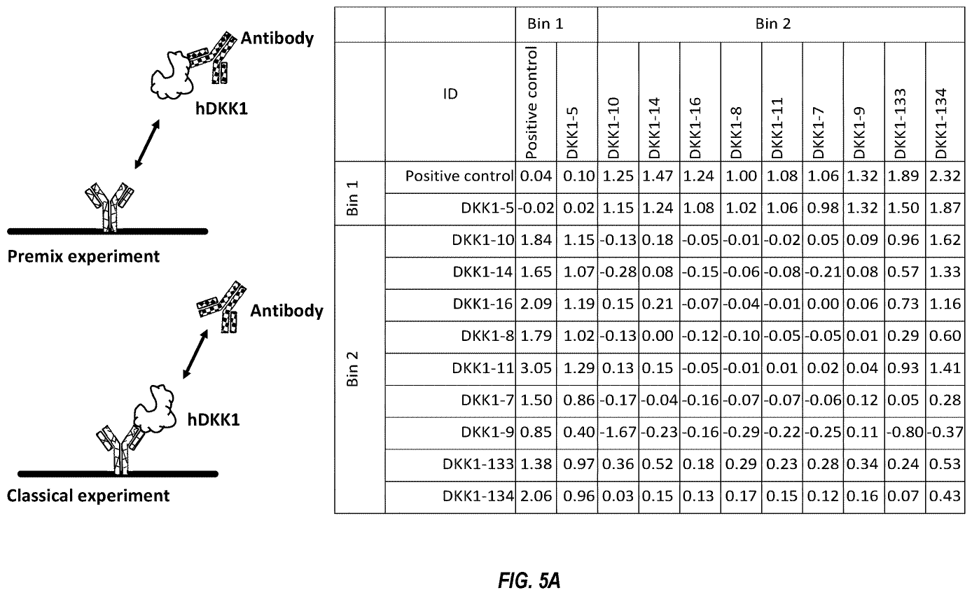

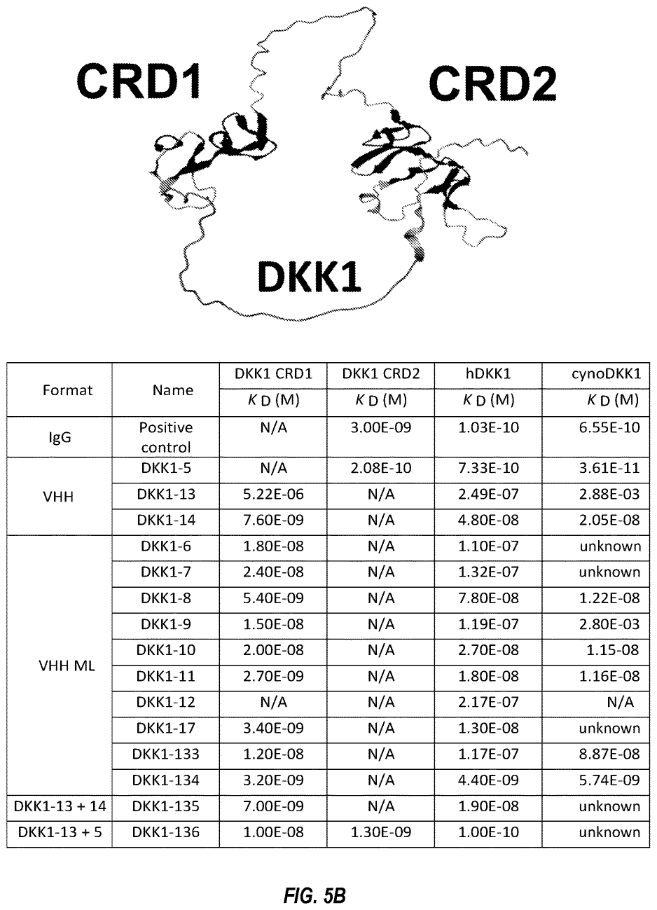

In this study, anti-DKK1 antibodies that block the binding of DKK1 to the receptor were generated from a nucleic acid library (FIGS. 4A-4B). Anti-DKK1 antibody binding to DKK1 was determined using surface plasmon resonance (SPR) analysis. Two epitope bins were apparent among DKK1 antibodies through epitope binning analysis. As shown in the antibody binding schematic to the left, formation of Ab-Ag-Ab complex indicates the antibodies are not binding to the same epitope of DKK1 (FIG. 5A). Two bins were identified. Anti-DKK1 antibodies were shown to bind to hDKK1 cysteine-rich domain (CRD)1 or CRD2, and several anti-DKK1 antibodies cross reacted with cynomolgus monkey DKK1 (cynoDKK1) (FIG. 5B). Epitope mapping studies confirmed binding of DKK1 binders to DKK1 CRD1 or CRD2 (data not shown).

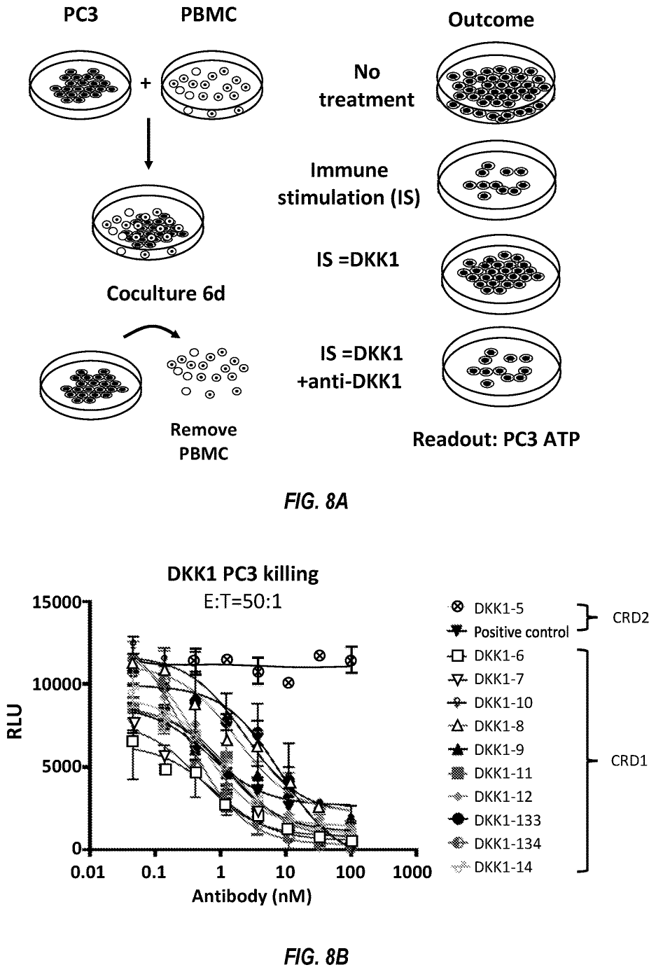

Binding of the antibodies to different cysteine-rich domains (CRDs) of human DKK1 (hDKK1) led to different activation effects. Three in vitro assays were used to assess antibody activity:
1) A Wnt TCF/LEF reporter assay to assess antibody effects on Wnt signaling.
2) An immune cell activation assay, in which human PBMCs are treated with immune stimulator, mWnt3a, hDKK1, and anti-DKK1 antibodies. Cytokine release of GM-CSF, markers for NK cell activation, is measured by ELISA.
3) In vitro PC3 tumor cytotoxicity by activated immune cells. Immune cells are activated and co-cultured with PC3 tumor cells for 6 days and PC3 killing is assessed. See FIG. 8A.

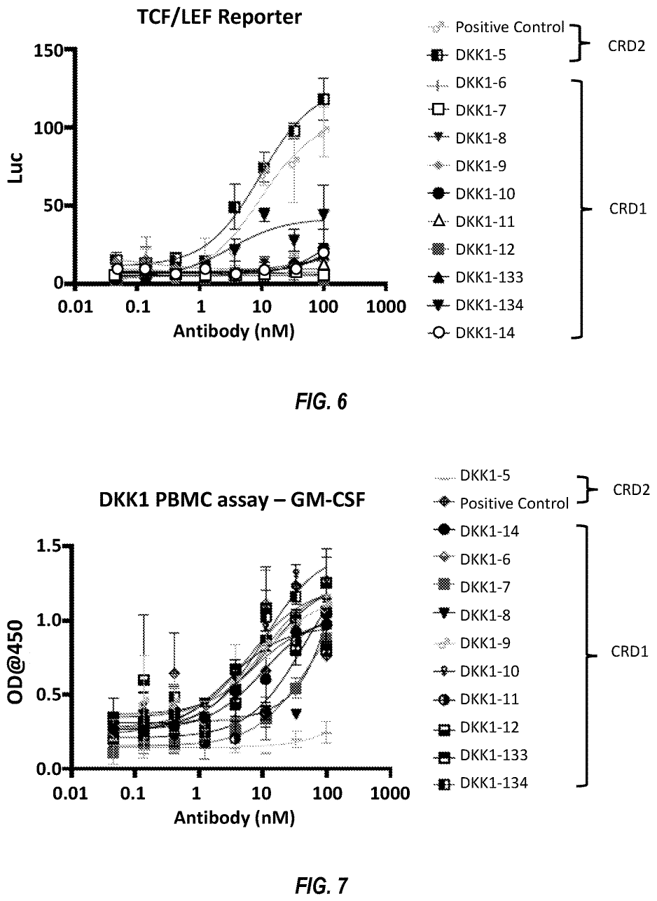

The in vitro functional assays showed that the interaction of Wnt to its receptor was restored in the presence of anti-DKK1 antibodies that bind to DKK1 CRD2, resulting in TCF/LEF signaling upregulation (FIG. 6). Moreover, anti-DKK1 antibodies binding to DKK1 CRD1 induced immune cell activation as measured by cytokine release (FIG. 7) and led to tumor cell cytotoxicity (FIGS. 8A-8B). DKK1-5 (DKK1-97 in WO 2023/091614), which binds to CRD2, did not appear to induce PC3 tumor cell cytotoxicity in this assay.

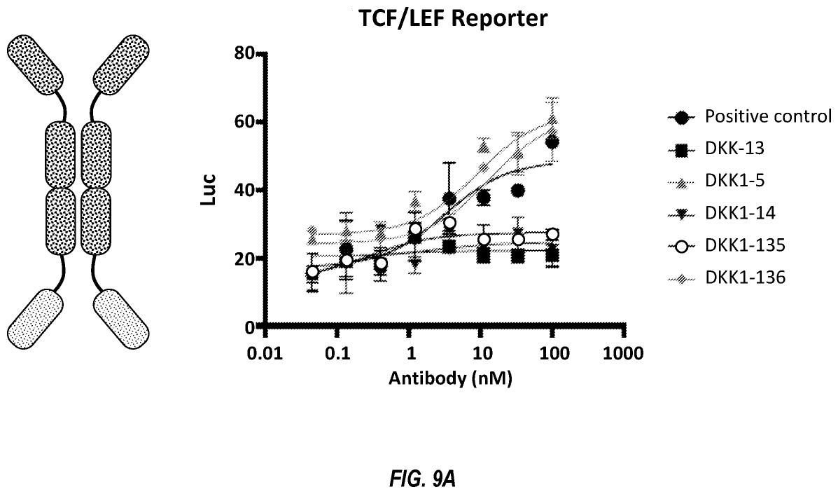

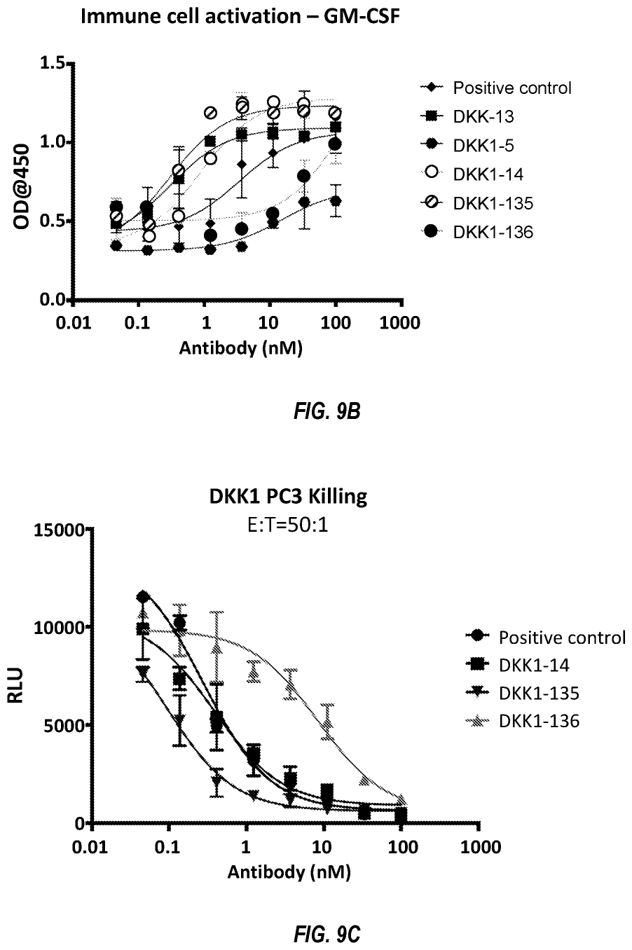

Bispecific formats (VHH-Fc region-VHH, FIG. 9A) were also tested in the Wnt TCF/LEF reporter assay (FIG. 9A), immune cell activation assay (FIG. 9B), and PC3 tumor cytotoxicity assay (FIG. 9C). DKK1-135 did not restore Wnt binding to the receptor, while DKK1-136 restored Wnt signaling in the TCF/LEF reporter assay (FIG. 9A). In contrast, DKK1-135 activated immune cells in the human PBMC assay and resulted in PC3 tumor cell cytotoxicity (FIGS. 9B-9C).

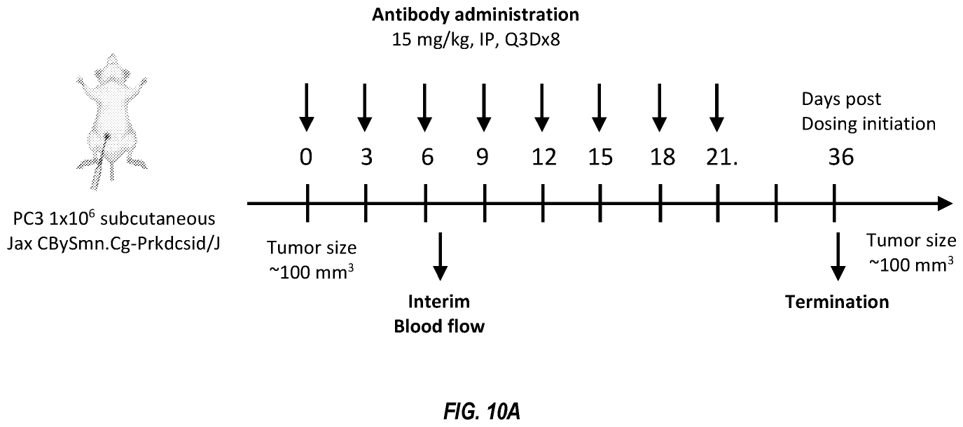

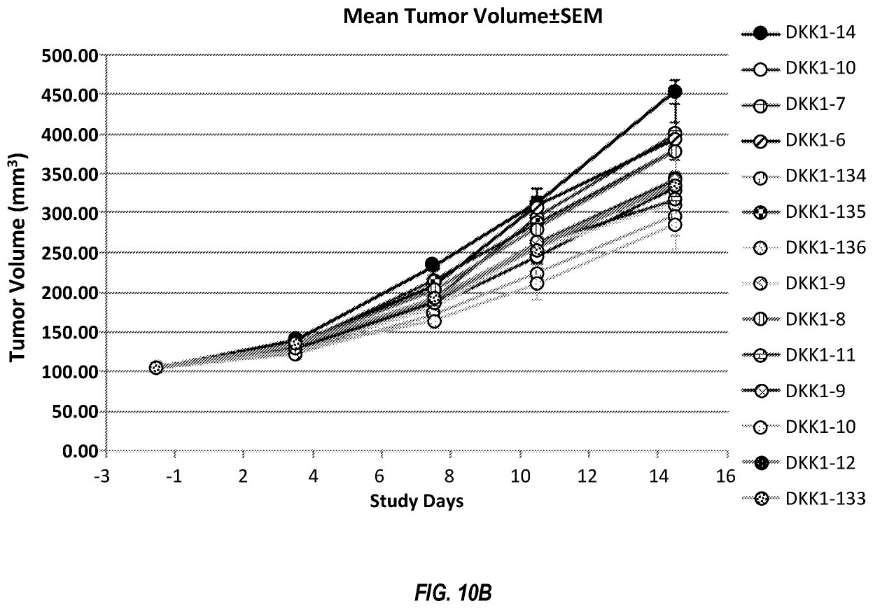

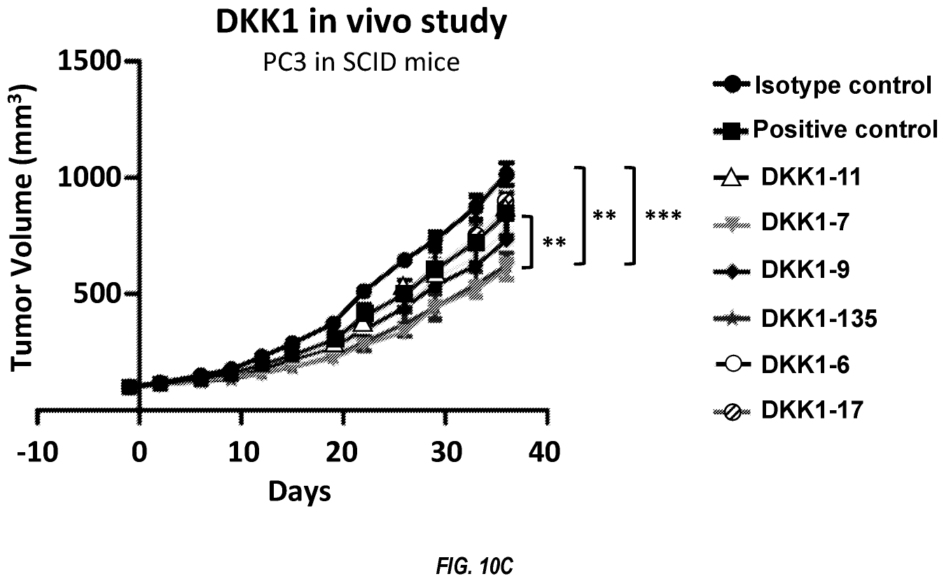

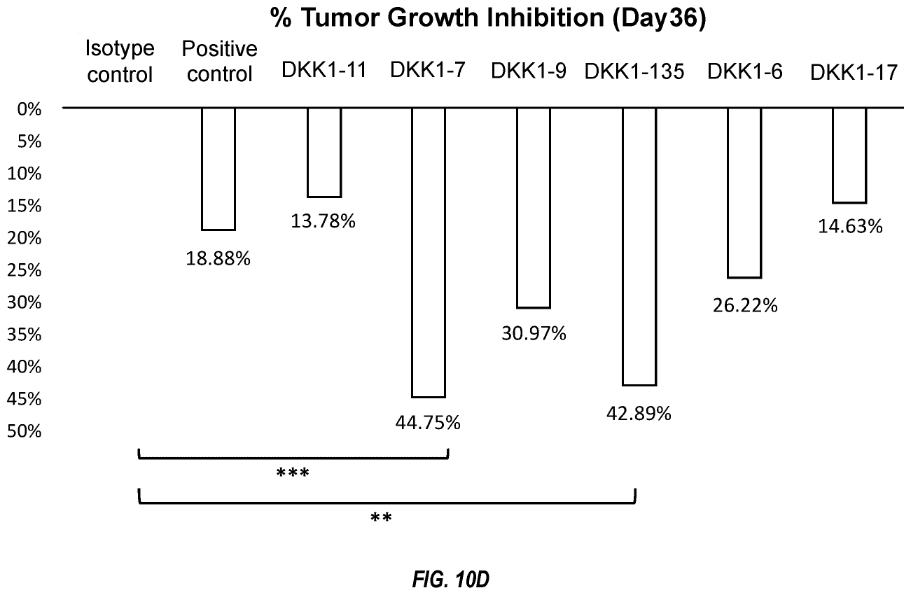

Various anti-DKK1 antibodies and bispecific constructs were tested in an in vivo tumor regression model (FIGS. 10C-10D). The anti-DKK1 antibodies led to tumor regression in the model (FIGS. 10A-10D). In vivo studies indicated anti-DKK1 antibodies and bispecific constructs promote tumor regression. Tumor size measurements indicate DKK1-7 and DKK1-135 treatment significantly suppress tumor growth. No significant difference was shown in body weight. **P 0.01, ***P 0.001 vs. isotype control by Kruskal-Walli's test (FIG. 10C). Tumor growth inhibition showed 44.75% and 42.89% inhibition upon DKK1-7 (DKK1-10 in in WO 2023/091614) and DKK1-135 (bispecific construct) treatment, respectively, while positive control showed only 18.88% inhibition (FIG. 10D). Toxicity study showed DKK1 binders described herein have little or no toxicity in blood panel and liver chemistry (data not shown).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A polypeptide that binds DKK1 comprising:
i) a first antibody variable domain comprising a CDR1 comprising the amino acid of SEQ ID NO: 11 or 17, a CDR2 comprising the amino acid sequence of SEQ ID NO:
12 or 18, and a CDR3 comprising the amino acid sequence of SEQ ID NO: 13 or 19; and
ii) a second antibody variable domain comprising a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

2. The polypeptide of claim 1, wherein:
a) (i) the first antibody variable domain comprises a CDR1 comprising the amino acid of SEQ ID NO: 11, a CDR2 comprising the amino acid sequence of SEQ ID NO: 12, and a CDR3 comprising the amino acid sequence of SEQ ID NO: 13; and (ii) the second antibody variable domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16; or
b) (i) the first antibody variable domain comprises a CDR1 comprising the amino acid of SEQ ID NO: 17, a CDR2 comprising the amino acid sequence of SEQ ID NO: 18, and a CDR3 comprising the amino acid sequence of SEQ ID NO: 19; and (ii) the second antibody variable domain comprises a CDR1 comprising the amino acid sequence of SEQ ID NO: 14, a CDR2 comprising the amino acid sequence of SEQ ID NO: 15, and a CDR3 comprising the amino acid sequence of SEQ ID NO: 16.

3. The polypeptide of claim 1, wherein:
(a) the first antibody variable domain comprises an amino acid sequence at least 90%, at least 95%, at least 96%, at least 97%, at least 98%, or at least 99% identical to the amino acid sequence of SEQ ID NO: 22 or SEQ ID NO: 23; and/or
(b) the first antibody variable domain comprises the amino acid sequence of SEQ ID NO: 22 or SEQ ID NO: 23.

4. (canceled)

5. The polypeptide of claim 1, wherein:
(a) the second antibody variable domain comprises an amino acid sequence at least 90%, at least 95%, at least 96%, at least 97%, at least 98%, or at least 99% identical to the amino acid sequence of the amino acid sequence of SEQ ID NO: 24;
(b) the second antibody variable domain comprises the amino acid sequence of SEQ ID NO: 24.

6. (canceled)

7. The polypeptide of claim 1, comprising an Fc region, wherein the Fc region is an IgG1, IgG2, IgG3, or IgG4 Fc region.

8. (canceled)

9. The polypeptide of claim 8, wherein the polypeptide has the structure: first antibody variable domain-linker-Fc region-linker-second antibody variable domain.

10. The polypeptide of claim 1, wherein the polypeptide comprises an amino acid sequence at least 90%, at least 95%, at least 96%, at least 97%, at least 98%, or at least 99% identical to the amino acid sequence of SEQ ID NO: 3 or SEQ ID NO: 4.

11. The polypeptide of claim 10, wherein the polypeptide comprises the amino acid sequence of SEQ ID NO: 3 or SEQ ID NO: 4.

12. A polypeptide that binds DKK1 comprising an antibody variable domain comprising a CDR1 comprising the amino acid sequence of SEQ ID NO: 5, a CDR2 comprising the amino acid sequence of SEQ ID NO: 6 or 29, and a CDR3 comprising the amino acid sequence of SEQ ID NO: 7 or 30.

13. (canceled)

14. The polypeptide of claim 12, wherein the polypeptide comprises the amino acid sequence of SEQ ID NO: 20.

15. The polypeptide of claim 12, comprising an Fc region, wherein the Fc region is an IgG1, IgG2, IgG3, or IgG4 Fc region.

16. (canceled)

17. (canceled)

18. The polypeptide of claim 12, wherein the polypeptide comprises the amino acid sequence of SEQ ID NO: 1.

19.-29. (canceled)

30. The polypeptide of claim 1, wherein the polypeptide binds to DKK1 with a K_{D} of less than 25 nM.

31. (canceled)

32. (canceled)

33. A pharmaceutical composition comprising the polypeptide of claim 1 and a pharmaceutically acceptable carrier.

34. An isolated nucleic acid that encodes the polypeptide of claim 1.

35. A vector comprising the nucleic acid of claim 34.

36. A host cell comprising the nucleic acid of claim 34.

37. A host cell that expresses the polypeptide of claim 1.

38. A method of producing the polypeptide of claim 1, comprising incubating the host cell of claim 36 under conditions suitable for expression of the polypeptide.

39. (canceled)

40. A method for treating a human subject having a cancer with elevated expression of DKK1 comprising administering to the subject a pharmaceutically effective amount of the polypeptide of claim 1.

41. (canceled)


SEQUENCE LISTING (30 sequences)

SEQ ID NO: 1 - protein, 348 aa, synthetic construct
EVQLVESGGGLVQPGGSLRLSCAASGRTFTMGWFRQAPGKERELVAAINRSGKSTYYADSVKGRFTISADNSKNTAYLQMNSLKPEDTAVYYCAAGNGGRHYGHSRARYDWGQGTLVTVSSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 2 - protein, 347 aa, synthetic construct
EVQLVESGGGLVQPGGSLRLSCAASGRGAMGWFRQAPGKEREFVAAINRSGKSTYYADSVKGRFTISADNSKNTAYLQMNSLKPEDTAVYYCAAGNGGRSYGHSRARYDWGQGTLVTVSSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 3 - protein, 490 aa, synthetic construct
EVQLVESGGGLVQPGGSLRLSCAASGRTFSNFAMGWFRQAPGKEREFVAVILRGGSTNYADSVKGRFTISADNSKNTAYLQMNSLKPEDTAVYYCAAAPRPKRVSVSYFSTASNYDWGQGTLVTVSSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSASEVQLVESGGGLVQPGGSLRLSCAASGRIFGGYAMGWFRQAPGKEREFVAAISWSGASAIYADSVKGRFTISADNSKNTAYLQMNSLKPEDTAVYYCAAGNGGSRYGHSRARYDWGQGTLVTVSS

SEQ ID NO: 4 - protein, 487 aa, synthetic construct
EVQLVESGGGLVQPGGSLRLSCAASGNIDRLYAMGWFRQAPGKEREFVAAISWSVSSTYYADSVKGRFTISADNSKNTAYLQMNSLKPEDTAVYYCAAEGGYSGTYYYTGDFDWGQGTLVTVSSGGGGSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGGGGGSGGGGSASEVQLVESGGGLVQPGGSLRLSCAASGRIFGGYAMGWFRQAPGKEREFVAAISWSGASAIYADSVKGRFTISADNSKNTAYLQMNSLKPEDTAVYYCAAGNGGSRYGHSRARYDWGQGTLVTVSS

SEQ ID NO: 5 - protein, 5 aa, synthetic construct
GRTFT

SEQ ID NO: 6 - protein, 8 aa, synthetic construct
INRSGKST

SEQ ID NO: 7 - protein, 17 aa, synthetic construct
AAGNGGRHYGHSRARYD

SEQ ID NO: 8 - skipped

SEQ ID NO: 9 - protein, 6 aa, synthetic construct
NRSGKS

SEQ ID NO: 10 - protein, 15 aa, synthetic construct
GNGGRSYGHSRARYD

SEQ ID NO: 11 - protein, 7 aa, synthetic construct
GRTFSNF

SEQ ID NO: 12 - protein, 5 aa, synthetic construct
LRGGS

SEQ ID NO: 13 - protein, 19 aa, synthetic construct
APRPKRVSVSYFSTASNYD

SEQ ID NO: 14 - protein, 7 aa, synthetic construct
GRIFGGY

SEQ ID NO: 15 - protein, 6 aa, synthetic construct
SWSGAS

SEQ ID NO: 16 - protein, 15 aa, synthetic construct
GNGGSRYGHSRARYD

SEQ ID NO: 17 - protein, 7 aa, synthetic construct
GNIDRLY

SEQ ID NO: 18 - protein, 6 aa, synthetic construct
SWSVSS

SEQ ID NO: 19 - protein, 15 aa, synthetic construct
EGGYSGTYYYTGDFD

SEQ ID NO: 20 - protein, 121 aa, synthetic construct
EVQLVESGGGLVQPGGSLRLSCAASGRTFTMGWFRQAPGKERELVAAINRSGKSTYYADSVKGRFTISADNSKNTAYLQMNSLKPEDTAVYYCAAGNGGRHYGHSRARYDWGQGTLVTVSS

SEQ ID NO: 21 - protein, 120 aa, synthetic construct
EVQLVESGGGLVQPGGSLRLSCAASGRGAMGWFRQAPGKEREFVAAINRSGKSTYYADSVKGRFTISADNSKNTAYLQMNSLKPEDTAVYYCAAGNGGRSYGHSRARYDWGQGTLVTVSS

SEQ ID NO: 22 - protein, 127 aa, synthetic construct
EVQLVESGGGLVQPGGSLRLSCAASGRTFSNFAMGWFRQAPGKEREFVAVILRGGSTNYADSVKGRFTISADNSKNTAYLQMNSLKPEDTAVYYCAAAPRPKRVSVSYFSTASNYDWGQGTLVTVSS

SEQ ID NO: 23 - protein, 124 aa, synthetic construct
EVQLVESGGGLVQPGGSLRLSCAASGNIDRLYAMGWFRQAPGKEREFVAAISWSVSSTYYADSVKGRFTISADNSKNTAYLQMNSLKPEDTAVYYCAAEGGYSGTYYYTGDFDWGQGTLVTVSS

SEQ ID NO: 24 - protein, 124 aa, synthetic construct
EVQLVESGGGLVQPGGSLRLSCAASGRIFGGYAMGWFRQAPGKEREFVAAISWSGASAIYADSVKGRFTISADNSKNTAYLQMNSLKPEDTAVYYCAAGNGGSRYGHSRARYDWGQGTLVTVSS

SEQ ID NO: 25 - protein, 266 aa, Homo sapiens
MMALGAAGATRVFVAMVAAALGGHPLLGVSATLNSVLNSNAIKNLPPPLGGAAGHPGSAVSAAPGILYPGGNKYQTIDNYQPYPCAEDEECGTDEYCASPTRGGDAGVQICLACRKRRKRCMRHAMCCPGNYCKNGICVSSDQNHFRGEIEETITESFGNDHSTLDGYSRRTTLSSKMYHTKGQEGSVCLRSSDCASGLCCARHFWSKICKPVLKEGQVCTKHRRKGSHGLEIFQRCYCGEGLSCRIQKDHHQASNSSRLHTCQRH

SEQ ID NO: 26 - protein, 222 aa, synthetic construct
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 27 - protein, 5 aa, synthetic construct
GGGGS

SEQ ID NO: 28 - protein, 12 aa, synthetic construct
GGGGSGGGGSAS

SEQ ID NO: 29 - protein, 6 aa, synthetic construct
NRSGKS

SEQ ID NO: 30 - protein, 15 aa, synthetic construct
GNGGRHYGHSRARYD